(19) United States
(12) Patent Application Publication
(10) Pub. No.: US 2008/0207895 A1
(43) Pub. Date: Aug. 28, 2008

(54) Methods for synthesizing polysaccharides

(76) Inventors: Robert D. Rosenberg, Staten Island, NY (US); Kuberan Balagurunathan, Salt Lake City, UT (US)

Correspondence Address: Pearl Cohen Zedek Latzer, LLP, 1500 Broadway, 12th Floor, New York, NY, 10036, US

(21) Appl. No.: 10/722,587

(22) Filed: Nov. 28, 2003

Related U.S. Application Data
(60) Provisional application No. 60/429,946, filed on Nov. 27, 2002.
(60) Provisional application No. 60/456,889, filed on Mar. 21, 2003.

(51) Int. Cl. C07H 3/00; C12P 19/04; G01N 33/00; C07H 1/06; C12Q 1/00

(52) U.S. Cl. 536/123.1; 435/101; 436/501; 435/4; 536/127

(57) ABSTRACT

This invention provides methods for the synthesis of HS polysaccharides or oligosaccharides. This invention provides HS polysaccharides and oligosaccharides thus obtained, and polysaccharides and oligosaccharides with anticoagulant activity.

### GOVERNMENT LICENSE RIGHTS

This invention was made with government support under Grant Numbers 1-P01-HL66105-01 and 5-P01-HL41484-12 awarded by the National Institutes of Health. The government has certain rights in the invention.

### FIELD OF THE INVENTION

The present invention relates generally to compositions and methods for synthesizing polysaccharides enzymatically. More particularly, invention relates to compositions and methods for synthesizing pentasaccharides enzymatically.

### BACKGROUND OF THE INVENTION

Polysaccharides interact with a number of proteins and regulate a wide variety of biological and pathological processes. For example, the heparan sulfate polysaccharide chains of heparan sulfate proteoglycans interact with growth factors, extracellular matrix components, protease inhibitors, proteases, lipoprotein lipase, and complement proteins. These interactions regulate the cell cycle, cell growth, cellular differentiation, cell proliferation, cell adhesion, cell migration, and lipid metabolism, as well as pathological phenomena such as inflammation, blood coagulation; and tumor cell invasion. In addition, pathogens such as bacteria, parasites, and viruses infect cells through their interactions with cell surface heparan sulfate.

Heparan sulfate (HS) is initially synthesized as a non-sulfated copolymer, attached to proteoglycan core proteins by sequential addition of D-glucuronic acid (GlcA) alternating with N-acetyl D-glucosamine (GlcNAc) residues, catalyzed by heparan sulfate polymerases. The polysaccharide chain then undergoes various modification steps, which include N-deacetylation and N-sulfation of GlcNAc, C-5 epimerase treatment of GlcA to L-iduronic acid (UdoA), 2-O sulfation of iduronic acid and 6-O sulfation and 3-O sulfation of glucosamine. Heparan sulfate is found ubiquitously on the surface of all cells, consists of domain structures that have extended unmodified regions separated by sulfated IdoA rich regions. By contrast, heparin, which is produced mainly by mast cells, consists predominantly of N- and O-sulfated, IdoA-rich sequences. All of the modification steps of heparin and heparan sulfate processing are catalyzed by different enzymes having different substrate specificities and occur in a sequential manner, such that the products in each step become substrates in subsequent steps. Heparan sulfate processing is selective in terms of the position and the number of modifications in a polysaccharide chain, leading to extensive structural and functional diversity.

Although many of the proteins that interact with heparan sulfate have been characterized, the polysaccharide structures that mediate these interactions have been identified in only a few cases (e.g., the heparan sulfate binding site on antithrombin III (AT-III) (FIG. 1), on glycoprotein D (gD) of herpes simplex virus I (FIG. 2), and on fibroblast growth factor 2 (FGF2) (not shown).

The synthesis or isolation of significant amounts of functional polysaccharides such as heparan sulfate is necessary for investigating the mechanisms of polysaccharide synthesis in vivo and their regulation of biological and pathological processes, for determining structure-function relationships between polysaccharides and their binding partners, for determining new biological or pathological targets, and for designing new or improved mimetics that interfere with or enhance binding of polysaccharides to their binding partners. The study of synthetic polysaccharides may provide information for redesigning currently-used drugs to eliminate side effects or other risk factors associated with use of the drug. For example, the animal-derived, poorly characterized polysaccharide heparin has been used for over seven decades as an anticoagulant in the treatment of stroke and myocardial infarction. However, animal-derived heparin can cause bleeding and heparin-induced thrombocytopenia (HIT) with arterial thrombosis, and its use may place a patient at risk of infection by animal-born pathogens, such as bovine spongiform encephalopathy.

Chemical synthesis of polysaccharides such as heparan sulfate can be used to design novel or non-natural structural motifs, as well as drugs with improved pharmacokinetic and pharmacodynamic characteristics. For example, chemically synthesized antithrombin III-binding heparan sulfate pentasaccharide has improved therapeutic action as compared to animal-derived heparin. However, there are several disadvantages to chemical synthesis of polysaccharides.

One disadvantage to the chemical synthesis of heparan sulfate is that it requires numerous steps, such as protection and deprotection steps. For example, the chemical synthesis of the antithrombin III-binding heparan sulfate pentasaccharide requires more than 50 steps. These methods are both time consuming and costly, and can preclude wide-spread clinical use of this anticoagulant, as well as the development of more effective anticoagulants of its class. In addition, the stereoselective glycosidic bond formation required for linkage of saccharide residues (e.g., glucosamine to glucuronic acid) is a daunting task despite the availability of several elegant glycosylation procedures. The formation of glycosidic linkage requires activation of a glycosyl donor to create reactive electrophilic species that couple with nucleophilic hydroxyl groups (acceptors). Such glycosidic linkages can occur either as alpha- or beta-anomers (e.g., in heparan sulfate glucosamine is α-glycosidically linked to adjacent glucuronic acid). In addition,-it is difficult to form the cis-glycosidic bond (α-linkage) with high specificity due to undesired anchimeric assistance from the C-2 group, leading to unwanted β-glycosidic linkage. Thus, masking of the amino function as an azido group or other non-participating group is required to generate alpha-linkage. In addition, the carboxyl group is generally masked as a protective hydroxyl group during glycosylation because the presence of this functional group renders GluA/IdoA as a poor glycosyl donor or acceptor. After completion of assembly of target polysaccharides, deprotected C-6 hydroxyl groups of glucose or idose are oxidized with a catalytic amount of 2,2,6,6,-tetramethylpiperidine-1-oxyl (TEMPO) and sodium hypochlorite as co-oxidant to regenerate carboxylic groups. In addition, uronic acids are prone to undergo epimerase treatment, which further complicates the synthesis, requiring additional protective group manipulations.

A need therefore exists for a rapid, efficient, and specific method for producing specially modified polysaccharides such as heparan sulfate or heparin-like polysaccharides.

### SUMMARY OF THE INVENTION

Cell-free, in vitro enzymatic and chemoenzymatic methods for synthesizing heparan sulfate/heparin-like polysaccharides have been developed. The synthesis of sufficient quantities of enzymatically modified polysaccharides permits the identification and study of their binding partners (e.g., proteins) and to use these interactions to identify critical groups that are central to the polysaccharide's biologic functions. The invention provides methods for designing mimetic drugs that contain these critical groups (e.g., in a non-carbohydrate framework). The invention also provides methods and reagents for studying polysaccharide biosynthesis and diversity, the enzymatic pathways that create particular modified polysaccharides, as well as the biological pathways in which the polysaccharides participate.

In one aspect, the invention provides methods for synthesizing quantities of bioactive polysaccharides such as heparan sulfate capable of binding to a binding partner. The invention provides methods of preparing a sulfated polysaccharide by treating an unsulfated or incompletely sulfated polysaccharide with at least one enzyme. In an embodiment, the invention provides methods of preparing heparan sulfate by treating an unsulfated heparan synthon or incompletely-sulfated heparan sulfate precursor with at least one enzyme. In another embodiment, the above methods require treatment of the unsulfated, incompletely sulfated polysaccharide, unsulfated heparan synthon, or incompletely-sulfated heparan sulfate precursor with at least one chemical reagent and at least one enzyme.

The chemical reagent may be an N-deacetylating reagent, an N-sulfating reagent, an epimerizing reagent, and/or an O-sulfating reagent. The enzyme may be an N-deacetylase, an N-sulfotransferase, an epimerase, and/or an O-sulfotransferase. In an embodiment, the method includes(a) treating an unsulfated polysaccharide such as a heparan synthon with an N-deacetylating reagent; (b) treating the step (a) product with an N-sulfating reagent; (c) treating the step (b) product with an epimerizing reagent; and (d) treating the step (c) product with at least one O-sulfating reagent. The order of the various steps can be varied.

The unsulfated polysaccharide may be isolated from a cell, such as a bacterial cell (e.g., E. coli). The deacetylating reagent may be a deacetylase, N-deacetylase-N-sulfotransferase, hydrazine: and/or a metal hydroxide. The N-sulfating reagent may be an N-sulfotransferase, N-deacetylase-N sulfotransferase, and/or trialkylamine sulfur trioxide. The epimerizing reagent may be C5-epimerase. The O-sulfating reagent may be a 3-O sulfotransferase, such as 3-OST1, 3-OST2, 3-OST3 (3-OST3a, 3-OST3b), 3-OST4, 3-OST5, and 3-OST6; a 6-O sulfotransferase, such as 6-OST1, 6-OST2a, 6-OST2b, and 6-OST3; or a 2-O sulfotransferase, such as 2-OST1.

In an embodiment, the method provides a combination of chemical and enzymatic synthesis. For example, non-sulfated N-acetyl heparosan K5, a capsular polysaccharide of E. coli bacterium, was used as an unsulfated heparan sulfate precursor or heparan synthon in the chemoenzymatic synthesis of bioactive heparan sulfate. In an embodiment, the method includes (1) isolation of an unsulfated heparan synthon; (2) N-deacetylase-N-sulfotransferase (NDST) treatment, or chemical N-deacetylation of the heparan synthon by either hydrazinolysis or alkaline treatment with 2M NaOH and N-sulfation with a chemoselective sulfating agent, such as trimethylamine sulfur trioxide, to form N-sulfated heparosan; (3) epimerase treatment of the N-sulfated heparosan with glucuronyl C-5 epimerase to make N-sulfated heparan sulfate; (4) 2-O sulfation of the N-sulfated heparan sulfate with 2-OST1 to make N-sulfated, 2-O sulfated heparan sulfate; (5) 6-O sulfation of the N-sulfated, 2-O sulfated heparan sulfate with 6-OST1 to make N-sulfated, 2-O sulfated, 6-O sulfated heparan sulfate; and (6) 3-O sulfation of the N-sulfated, 2-O sulfated, 6-O sulfated heparan sulfate with 3-OST1 to make N-sulfated, 2-O sulfated, 6-O sulfated, 3-O sulfated heparan sulfate, which is an active anticoagulant capable of binding to antithrombin III and inhibiting factor Xa and possibly other enzymes involved in blood coagulation. The order or the enzymatic steps (2)-(6) of the above reactions can be varied to produce the ultimate polysaccharide described in step (6).

In another embodiment, the methods of the invention can be enzymatic only. In this embodiment, the chemical N-deacetylation and N-sulfation of the heparan synthon is replaced with enzymatic treatment of the heparan synthon with N-deacetylase-N-sulfotransferase (NDST), which catalyses the two initial modifications of the polysaccharide precursor (N-deacetylation and N-sulfation) enzymatically.

In another embodiment, the invention provides methods for synthesizing a heparan sulfate that is not 2-O sulfated. In an embodiment, the method includes (1) isolation of an unsulfated heparan synthon; (2) N-deacetylase-N-sulfotransferase (NDST) treatment, or chemical N-deacetylation of the heparan synthon by either hydrazinolysis or alkaline treatment with 2M NaOH and N-sulfation with a chemoselective sulfating agent, such as trimethylamine sulfur trioxide, to form N-sulfated heparosan; (3) epimerase treatment of the N-sulfated heparosan with glucuronyl C-5 epimerase to make N-sulfated heparan sulfate; (4) 6-O sulfation of the N-sulfated heparan sulfate with 6-OST1 to make N-sulfated, 6-O sulfated heparan sulfate; and (5) 3-O sulfation of the N-sulfated, 6-O sulfated heparan sulfate with 3-OST1 to make N-sulfated, 6-O sulfated, 3-O sulfated heparan sulfate, which is an active anticoagulant capable of binding to antithrombin III but does not bind to platelet factor 4. It is also possible to carry out 3-O sulfation at any stage in generating the bioactive structure.

For example, the order of 3-O and 6-O -sulfation could be switched to generate the bioactive structure. The order or the enzymatic steps (2)-(5) of the above reactions can be varied to produce the ultimate polysaccharide described in step (5).

In another embodiment, the invention provides methods for synthesizing a heparan sulfate that is 3-O sulfated by isoform 3-OST3a such that it binds to herpes simplex virus 1 glycoprotein D (HSV-gD). In an embodiment, the method includes (1) isolation of an unsulfated heparan synthon; (2) N-deacetylase-N-sulfotransferase (NDST) treatment, or chemical N-deacetylation of the heparan synthon by either hydrazinolysis or alkaline treatment with 2M NaOH and N-sulfation with a chemoselective sulfating agent, such as trimethylamine sulfur trioxide, to form N-sulfated heparosan; (3) epimerase treatment of the N-sulfated heparosan with glucuronyl C-5 epimerase to make N-sulfated heparan sulfate; (4) 2-O sulfation of the N-sulfated heparan sulfate with 2-OST1 to make N-sulfated, 2-O sulfated heparan sulfate; (5) 6-O sulfation of the N-sulfated, 2-O sulfated heparan sulfate with 6-OST1 to make N-sulfated, 2-O sulfated, 6-O sulfated heparan sulfate; and (6) 3-O sulfation of the N-sulfated, 2-O sulfated, 6-O sulfated heparan sulfate with 3-OST3a to make N-sulfated, 2-O sulfated, 6-O sulfated, 3-O sulfated heparan sulfate, which binds to HSV-gD. The order or the enzymatic steps (2)-(6) of the above reactions can be varied to produce the ultimate polysaccharide described in step (6).

In another embodiment, the invention provides methods for synthesizing a heparan sulfate pentasaccharide that binds to ATIII. In an embodiment, the method includes (1) isolation of an unsulfated heparan synthon polysaccharide; (2) N-deacetylase-N-sulfotransferase (NDST) (e.g., NDST1, NDST2, NDST3, and/or NDST4) treatment to form partially N-deacetylated, N-sulfated heparosan polysaccharide; (3) partial heparitinase treatment of the partially N-deacetylated, N-sulfated heparosan polysaccharide by digestion with heparitinase (e.g., heparitinase I, I, and/or III) to make a NDST-treated, heparitinase-cleaved, hexasaccharide; (4) epimerization and 2-O sulfation of the NDST-treated, heparitinase-cleaved, hexasaccharide with an epimerase and 2-OST1 to make an epimerized, 2-O-sulfated, NDST-treated, heparitinase-cleaved, hexasaccharide; (5) 6-O sulfation of the epimerized, 2-O-sulfated, NDST-treated, heparitinase-cleaved, hexasaccharide with 6-OST1 and either 6-OST2a, 6-OST2b, or 6-OST3 to make 6-O sulfated, epimerized, 2-O-sulfated, NDST-treated, heparitinase-cleaved hexasaccharide; (6) Δ^{4,5} glycuronidase treatment of the 6-O sulfated, epimerized, 2-O-sulfated, NDST-treated, heparitinase-cleaved, hexasaccharide to make Δ^{4,5} glycuronidase-treated, 6-O sulfated, epimerized, 2-O-sulfated, NDST-treated, heparitinase-cleaved, pentasaccharide; and (7) 3-O sulfation of the Δ^{4,5} glycuronidase-treated, 6-O sulfated, epimerized, 2-O-sulfated, NDST-treated, heparitinase-cleaved pentasaccharide with 3-OST1, 3-OST5, and/or 3-OST6 to make a 3-O sulfated, Δ^{4,5} glycuronidase-treated, 6-O sulfated, epimerized, 2-O-sulfated, NDST-treated, heparitinase-cleaved pentasaccharide, which binds to AT-III. The order or the enzymatic steps (2)-(7) of the above reactions can be varied to produce the-ultimate pentasaccharide described in step (7).

Any of the steps of the enzymatic syntheses described herein may be performed in any number of orders, as desired. In addition, a skilled artisan my use functional analogues or derivatives of polysaccharides or oligosaccharide to perform the methods of the invention to obtain functionally equivalent molecules or molecules with altered binding capabilities or specificities. It is further contemplated that each combination of enzymatic reactions described herein, in any order, as well as portions of the described synthesis reactions comprising any number of steps, as well as each step of each synthesis reaction provide embodiments of the invention.

In another embodiment, the invention provides a method for determining the structural identification of the hexasaccharide derived from the partial heparitinase treatment of the partially N-deacetylated, N-sulfated heparosan polysaccharide by treatment with Δ^{4,5}-β-glycuronidase and α-N-Acetylglucosaminidase. This method is useful for monitoring the synthesis of the partially heparitinase-treated, partially N-deacetylated, N-sulfated heparosan hexasaccharide. The order of the Δ^{4,5}-β-glycuronidase and α-N-Acetylglucosaminidase treatments may vary.

In another aspect, the invention provides synthetic polysaccharides, such as heparan sulfate, capable of binding to antithrombin III or HSV-gD. In an embodiment, the invention provides a heparan sulfate that is N-sulfated, epimerized, 2-O sulfated, 6-O sulfated, and 3-O sulfated and is capable of binding to antithrombin III. In another embodiment, the invention provides an improved antithrombin III-binding heparan sulfate that is N-sulfated, epimerized, 6-O sulfated, and 3-O sulfated (i.e., is not 2-O-sulfated). In another embodiment, the invention provides a heparan sulfate that is N-sulfated, epimerized, 2-O sulfated, 6-O sulfated, and 3-O sulfated with a 3-OST3a isoform and is capable of binding to HSV-gD.

In another aspect, the invention provides methods for detecting or identifying polysaccharide-binding ligands by screening various ligand libraries against a modified polysaccharide under suitable conditions and detecting or measuring the interaction. For example, a phage display library or secretion library may be used in conjunction with gel mobility shift assays (GMSAs), surface plasmon resonance (SPR), or mass spectrometry.

In another aspect, the invention provides methods for identifying the enzymes that are required for the synthesis of bioactive polysaccharides. For example, 3-OST1 or 3-OST3a-modified polysaccharides can be tested for their ability to generate anticoagulant or antiviral structures by carrying out bioassays such as a factor Xa assay or a viral entry assay, respectively. In an embodiment, the invention provides methods for elucidating the enzymatic pathway for a given modified polysaccharide by determining the identity and order of action of the enzymes required for synthesizing the modified polysaccharide.

In another aspect, the invention provides methods for identifying critical groups on polysaccharides that are required for binding to a binding partner. If a given enzyme is necessary and sufficient to generate a bioactive structure, then this enzyme isoform defines the critical group which can be identified within heparan sulfate structures by using a stable isotope containing sulfate in conjunction with mass spectrometry or radioactive sulfate with GMSA.

### BRIEF DESCRIPTION OF THE DRAWINGS

The foregoing and other objects, features and advantages of the present invention, as well as the invention itself, will be more fully understood from the following description of preferred embodiments when read together with the accompanying drawings, in which:

FIG. 1 illustrates an antithrombin III-binding pentasaccharide heparan sulfate structure. A, glucosamine; B, glucuronic acid; C, glucosamine; D, iduronic acid; E, glucosamine; R, Acetyl (Ac) or sulfate (SO₃⁻).

FIG. 2 illustrates a putative gD-binding tetramer heparan sulfate structure. A, glucosamine; B, iduronic acid; C, glucosamine; D, glu/iduronic acid.

FIG. 3 illustrates the biosynthetic modifications that take place in heparan sulfate biosynthesis. Polysaccharide 1 is a heparan synthon. Polysaccharide 2 is an N-sulfated heparosan formed after chemical N-deacetylation of the heparan synthon with either hydrazinolysis or alkaline treatment with 2M NaOH and N-sulfation with trimethylamine sulfur trioxide. Polysaccharide 3 is N-sulfated heparin sulfate or heparin after epimerase treatment of the N-sulfated heparosan with glucuronyl C-5 epimerase. Polysaccharide 4 is N-sulfated, 2-O sulfated-heparan sulfate, after 2-O sulfation of the N-sulfated heparan sulfate with 2-OST1. Polysaccharide 5 is N-sulfated, 2-O sulfated, 6-O sulfated heparan sulfate, after 6-O sulfation of the N-sulfated, 2-O sulfated heparan sulfate with 6-OST1. Polysaccharide 6 is N-sulfated, 2-O sulfated, 6-O sulfated, 3-O sulfated heparan sulfate, after 3-O sulfation of the N-sulfated, 2-O sulfated, 6-O sulfated heparan sulfate with 3-OST1.

FIG. 4 illustrates the biosynthetic modifications that take place in the biosynthesis of a heparan sulfate molecule that lacks O-sulfation. Polysaccharide 3 is N-sulfated heparin sulfate or heparin. Polysaccharide 7 is N-sulfated. 6-O sulfated heparan sulfate, after 6-O sulfation of the N-sulfated heparan sulfate with 6-OST1. Polysaccharide 8 is N-sulfated, 6-O sulfated, 3-O sulfated heparan sulfate, after 3-O sulfation of the N-sulfated, 6-O sulfated heparan sulfate with 3-OST1.

FIG. 5 illustrates the biosynthetic modifications that take place in the biosynthesis of a heparan sulfate molecule with binding specificity for glycoprotein D (gD) of Herpes Simplex virus (HSV) after it is 3-O sulfated with 3-OST3a. Polysaccharide 5 is N-sulfated, 2-O sulfated, 6-O sulfated heparan sulfate, after 6-O sulfation of the N-sulfated, 2-O sulfated heparan sulfate with 6-OST1. Polysaccharide 9 is N-sulfated, 2-O sulfated, 6-O sulfated, 3-O sulfated heparan sulfate, after 3-O sulfation of the N-sulfated, 2-O sulfated, 6-O sulfated heparan sulfate with the 3-OST isoform 3-OST3a.

FIG. 6 illustrates the human C5 epimerase expression vector pXEPIM.

FIG. 7 illustrates a gel mobility shift assay of the antithrombin III-binding heparan sulfate polysaccharide with and without 3-O sulfation. (A) Polysaccharide alone, lacking 3-O sulfate; (B) Polysaccharide lacking 3-O sulfate and antithrombin III; (C) Polysaccharide alone, containing 3-O sulfate; (D) Polysaccharide containing 3-O sulfate and antithrombin III.

FIG. 8 illustrates the mechanism of anticoagulant action of the antithrombin III-binding heparan sulfate pentasaccharide. The pentasaccharide binds to antithrombin III, which is then able to bind to Factor Xa to form an anticoagulant complex. The pentasaccharide is recycled for further binding to antithrombin III.

FIG. 9 illustrates the treatment of Polysaccharide 2 with epimerase, 6-OST and nitrous/sodium borohydride to obtain disaccharides GlcA-anMan_{R}6S and IdoA-anMan_{R}6S. Polysaccharide 2 is an N-sulfated heparosan formed after chemical N-deacetylation of the heparan synthon with either hydrazinolysis or alkaline treatment with 2M NaOH and N-sulfation with trimethylamine sulfur trioxide. Polysaccharide 3 is an epimerized, N-sulfated heparin sulfate or heparin after epimerase treatment of the N-sulfated heparosan with glucuronyl C-5 epimerase. Polysaccharide 7 is N-sulfated, epimerized, 6-O sulfated heparan sulfate, after 6-O sulfation of the N-sulfated, epimerized, heparan sulfate with 6-OST1. GlcA-anMan_{R}6S and IdoA-anMan_{R}6S are the disaccharide products of nitrous/sodium borohydride treatment of the N-sulfated, 6-O sulfated heparan sulfate.

FIG. 10 illustrates an HPLC chromatogram of the disaccharide of nitrous/sodium borohydride treatment of the N-sulfated, epimerized, 6-O sulfated heparan sulfate. Arrow 1 indicates radioactive SO₄⁻². Arrow 2 indicates GlcA-anMan_{R}6S. Arrow 3 indicates IdoA-anMan_{R}6S.

FIG. 11 illustrates the selective incorporation of sulfate by 2-OST1 into Polysaccharide 3, showing a 5% polyacrylamide gel in which Lane 1 contains 2-OST1 treated non-epimerized, N-sulfated, heparan sulfate and Lane 2 shows an epimerized, N-sulfated, 2-O sulfated heparan sulfate, after epimerization and 2-O sulfation of the N-sulfated heparan sulfate (Polysaccharide 4).

FIG. 12 illustrates an HPLC chromatogram of heparitinase (I, II and III) digested Polysaccharide 7, an N-sulfated, 6-O sulfated heparan sulfate, after 6-O sulfation of the N-sulfated heparan sulfate (Polysaccharide 3) with 6-OST1 using PAP³⁵S.

FIG. 13 illustrates an HPLC chromatogram of heparitinase (I, II and III) digested Polysaccharide 8, an N-sulfated, 6-O sulfated, 3-O-sulfated heparan sulfate, after 3-O sulfation of the N-sulfated, 6-O sulfated heparan sulfate (Polysaccharide 7) with 3-OST1 using PAP³⁵S.

FIG. 14 illustrates extractive ion chromatography (XIC) of the trisulfated species obtained from heparitinase (I, II and III) digestion of Polysaccharide 6, wherein FIG. 14A illustrates the XIC of Polysaccharide 6, FIG. 14B illustrates the electrospray mass spectra of peak A of FIG. 14A, and FIG. 14C illustrates the electrospray mass spectra of peak B of FIG. 14A.

FIG. 15 illustrates extractive ion chromatography (XIC) of the trisulfated species obtained from heparitinase (I, II and III) digestion of Polysaccharide 8, wherein FIG. 15A illustrates the XIC of Polysaccharide 8 and FIG. 15B illustrates the electrospray mass spectra of Polysaccharide 8.

FIG. 16 illustrates the structural identification of the hexasaccharide 11 by treatment with exo-enzymes Δ^{4,5}-β-glycuronidase-and α-N-Acetylglucosaminidase, forming Polysaccharide 16 and Polysaccharide 17, respectively.

FIG. 17 illustrates the electrospray mass spectra profile of Hexasaccharide 11.

FIG. 18 illustrates the electrospray mass spectra profile of Pentasaccharide 16.

FIG. 19 illustrates the electrospray mass spectra profile of Pentasaccharide 17.

FIG. 20 illustrates the biosynthetic modifications in the biosynthesis of Antithrombin III binding Pentasaccharide 15.

FIG. 21 illustrates the electrospray mass spectra profile of Hexasaccharide 12.

FIG. 22 illustrates the electrospray mass spectra profile of Hexasaccharide 13.

FIG. 23 illustrates the heparatinase cleavage sites of Hexasaccharide 13 and biosynthetic modifications when Hexasaccharide 13 is cleaved by heparitinases I, II, and III to obtain disaccharides.

FIG. 24 illustrates the electrospray mass spectra profile of Polysaccharide 13.

FIG. 25 illustrates the mass spectra analysis of Pentasaccharide 15, wherein FIG. 25A illustrates the XIC of Pentasaccharide 15 and FIG. 25B illustrates the electrospray mass spectra of Pentasaccharide 15. Pentasaccharide 15 contains critical 3-O ³⁴SO₄ group as a mass spectral probe and isotope clusters of parent ion is illustrated as insets.

FIG. 26 illustrates a gel mobility shift assay of radiolabeled heparan sulfate with the 6-OST1 or 3-OST1 isoform. In each lane, about 50,000 cpm Polysaccharide 5 (panel a), Polysaccharide 6 (panel b), Polysaccharide 8 (panel c), Pentasaccharide 15 (panel d) were incubated with or without 1 μg of AT-III were loaded and resolved in a 5% native PAGE gel.

### DETAILED DESCRIPTION OF THE INVENTION

As used herein, the term “binding partner” refers to any molecule that binds to a polysaccharide and can include a protein, peptide, polysaccharide, antibody, etc.

As used herein, the term “critical group” or “functional group” or the like refers to a specific atom or a group of atoms that, when present in a biomolecule at a certain position on the biomolecule, provides the biomolecule with at least a specific chemical function or characteristic (e.g., for example, a binding characteristic, enzymatic activity, or other functional activity). A critical group may work in concert with, or interact with, another critical group(s) to provide the biomolecule with the specific chemical function. Exemplary critical groups include sulfate groups, carboxylate groups, hydroxyl/amino groups, and acetyl groups.

As used herein, the terms “gel mobility shift assay” or “GMSA” refer to the use of gel electrophoresis, e.g., polyacrylamide gel electrophoresis (PAGE), to determine band retardation or gel shift and, optionally, may refer to the use of autoradiography, if a radioactive probe is used. However, the invention contemplates the use of any other binding assay that will detect a difference in the amount or size of a labeled polysaccharide and/or binding partner that is bound to a binding partner and/or polysaccharide, respectively, and a labeled polysaccharide and/or binding partner that is not bound to a binding partner and/or polysaccharide, respectively. Other binding assays include but are not limited to capillary electrophoresis or capillary HPLC.

As used herein, the term “isolated”, when referring to a polysaccharide, binding partner or other component of the instant invention, means that the component has been removed from its natural environment, e.g., the body (i.e., it is derived from the natural source).

As used herein, the terms “modify” or “modified” refer to the modification of polysaccharides by chemicals or enzymes such as sulfotransferases (e.g., NDSTs) and epimerases. Such modification may occur in vivo, e.g., in the Golgi apparatus or in any other location in the cell, or outside the cell such that functional groups, or their precursors or intermediates, are added, altered, or removed from the polysaccharide. In another embodiment, the modification of the polysaccharide may occur in vitro.

As used herein, the term “polysaccharide” is used to refer to a polymer of glycosidically-linked saccharide residues, as well as chemically modified analogues or derivatives that may be produced by well known methods in the art. Although heparan sulfate is used as an exemplary polysaccharide herein, many other polysaccharides that bind to a binding partner may be used or studied in the instant invention. Such other polysaccharides include, but are not limited to, heparin, hyaluronic acid, chondroitin sulfate, dermatan sulfate, and keratan sulfate, or similar molecules or hybrids of the above structures. The basic disaccharide repeats of heparan sulfates, chondroitin sulfates, dermatan sulfates, hyaluronic acids, and keratan sulfates are well known in the art. For example, heparin sulfate is composed of alternating N-acetyl or N-sulfo D-glucosamine (GlcNAc/NS) and D-glucuronic acid (GlcA) or IdoA. A pentamer of heparan sulfate may comprise GlcNAc/NS-GlcA/IdoA-GlcNS-GlcA/IdoA-GCcNS or may comprise GlcA/IdoA-GlcNAc/NS-GlcA/IdoA-GlcNS-GlcA/IdoA. The composition of chondroitin sulfate is (GalNAc-GlcA)ₙ. The composition of dermatan sulfate is (GalNAc-GlcA/IdoA)ₙ. The composition of keratan sulfate is (Gal-GlcNAc)ₙ. The polysaccharides of the invention may be of variable length, e.g., from about 20-300 saccharides or longer. The composition of the polysaccharide depends upon the binding partner, or application of the invention.

Binding partners may be prepared according to art known methods such as bacterial expression and protein purification, in vitro transcription and translation, peptide synthesis, monoclonal or polyclonal antibody preparation, affinity purification of proteins, etc.

Gel mobility shift assay (GMSA) can be used to analyze the binding of a polysaccharide with a binding partner. For example, heparan sulfate polysaccharide and binding partner antithrombin-III may be used to characterize the functional groups required for binding of heparan sulfate to antithrombin-III or heparan sulfate to gD.

### Preparation of an N-sulfated, 2-O Sulfated, 6-O Sulfated, 3-O Sulfated Heparan Sulfate Polysaccharide

Preparation of an unsulfated polysaccharide precursor: An unsulfated polysaccharide precursor or synthon, such as a heparan sulfate precursor (heparan synthon) is chemically synthesized by standard glycosylation methods (Toshima and Tatsuta (1993) Chem. Rev. 93:1503-31). For example, a glycosylation method using aziosugar having bromide groups as a glycosyl donor is as follows. Briefly, a suspension of an equimolar amount of glycosyl acceptor and 2-azido glycosyl bromide (glycosyl donor) and activated powdered 4 Å molecular sieves in a 1:1 mixture of dry CH₂Cl₂/benzene is stirred for 1 hour at room temperature under a dry argon atmosphere. The mixture is cooled to 40° C. and AgClO₄ (1 mmole) s added. The reaction is slowly brought to room temperature over 3 hours and stirred overnight. The reaction mixture is diluted with methylene chloride, filtered through celite, washed with water, and dried over anhydrous Na₂SO₄. The organic layer is concentrated by rotary evaporation and purified by silica flash column chromatography to yield the target oligosaccharides. (Lemieux et al. (1979) Can. J. Chem. 57:1244-51).

Another example of a glycosylation method in which thiophenyl glycosides are used as the glycosyl donor is as follows. A solution of glycosyl donor (thiopheyl glycoside) and glucopyranoside (glycosyl acceptor) in dry dichloromethane containing 4 Å molecular sieves, is stirred for 30 minutes at room temperature and then cooled to 0° C. N-iodosuccinimide and trifluoromethanesulfonic acid are added, and the mixture is allowed to reach room temperature. After 1 hour, saturated aqueous NaHCO₃ is added, and the mixture is filtered through celite. The filtrate is diluted with dichloromethane and the solution is washed with 10% aqueous Na₂S₂O₃, water, and concentrated. Column chromatography is used to yield pure target compound. (Kovensky et al. (1999) Bioorg Med Chem. 7:1567-80).

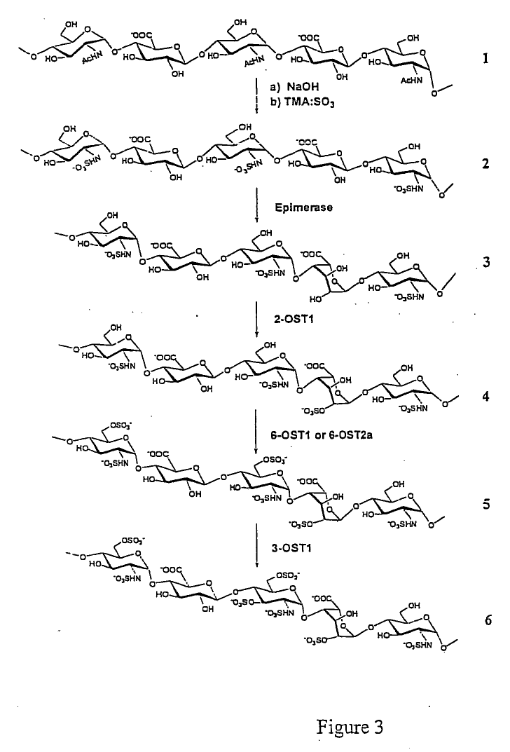

Alternatively, the unsulfated polysaccharide precursor is isolated from a cell, such as a bacterial cell as described, for example, in Example 2. Isolated polysaccharide is partially digested with heparitinase enzymes and subjected to capillary reverse phase ion-pair high performance liquid chromatography coupled to micro-electrospray ionization time of flight mass spectroscopy (“RP-IP-HPLC-ESI-TOF-MS” or “LC/MS”) (Kuberan et al. (2002) J. Am. Chem. Soc. 124:8707-18) (Example 7). An exemplary heparan synthon is Polysaccharide 1 shown in FIG. 3.

The polysaccharide of the instant invention may be heparan sulfate or another glycosaminoglycan, such as heparin and hyaluronic acid, a galactosaminoglycan, such as e.g., chondroitin-4-sulfate, chondroitin-6-sulfate, and dermatan sulfate, or a sulfated polylactosamine, such as, e.g., keratan sulfate (KS) or hybrids thereof.

Chemical N-Deacetylation and N-Sulfation of the Polysaccharide: Step two of the method involves chemical alteration of the polysaccharide synthon to make an N-deacetylated and N-sulfated polysaccharide, such as N-sulfated heparosan. N-deacetylation may be accomplished by hydrazinolysis at 90-100° C. (Shaklee et al. (1984) Biochem. J. 217:187-97). Alternatively, the polysaccharide synthon is N-deacetylated and N-sulfated by alkaline treatment of the polysaccharide synthon with 2M NaOH at 60-65° C. overnight (Leali et al. (2001) J. Biol. Chem. 276:37900-08). In short, one gram of polysaccharide is dissolved in 100 ml of 2.0N NaOH, incubated for 24 hours at 60° C., cooled to room temperature, and the pH adjusted to 7.0. The solution is warmed up to 40° C., added in a single step to 1.6 g of sodium carbonate and 1.6 g of pyridine-sulfotrioxyde complex stepwise for 4 hours, and incubated for an additional hour at the same temperature. The solution is then brought to room temperature, and the pH adjusted to 7.5-8.0. The N-deacetylated/N-sulfated polysaccharide is purified from salts by ultracentrifugation, and the sample dried under a vacuum. Either of these methods results in complete removal of acetyl groups.

The resulting free amino groups exposed by the N-deacetylation are selectively N-sulfated using chemoselective sulfating agents such as trimethylamine sulfur trioxide under careful reaction conditions to make an N-sulfated polysaccharide such as N-sulfated heparosan (FIG. 3, Polysaccharide 2) (Example 5). N-deacetylation and N-sulfation is confirmed by partial digestion of the N-sulfated polysaccharide using heparitinases and capillary IP-RP-HPLC-micro ESI-TOF-MS analysis (Example 7). Mass spectrometric analysis demonstrates the presence of anticipated disaccharides with a molecular weight of 417 Daltons.

An alternative but more complex enzymatic approach to the N-deacetylation and N-sulfation step requires N-deacetylase-N-sulfotransferase, which catalyzes the N-deacetylation and N-sulfation modifications of the polysaccharide precursor (Aikawa et al. (2001) J. Biol. Chem. 276:5876-82). For N-deacetylation and N-sulfation, a polysaccharide precursor is incubated with 50 mM MES, pH 6.5, containing 1% Triton X-100, 10 mM MnCl₂, and 25-50 ng of N-deacetylase-N-sulfotransferase. After 30 min. at 37° C., the reaction is stopped by the addition of 0.5 volumes of 0.2 M HCl 1 volume of 0.1 M acetic acid, and 1 volume of water. Acetic acid is recovered by extracting the sample three times with 1 volume of ethyl acetate and an aliquot of the pooled fractions (0.5 ml) are analyzed by liquid scintillation. For N-sulfation, 10 μM PAPS is incubated for 1 hour at 37° C. in 50 mM HEPES, pH 7.0, 1% Triton X-100, 10 mM MgCl₂, 1 mM MnCl₂, 25 μg heparosan or N-desulfated heparin, in a total volume of 50 μl. The reaction is stopped by the addition of 2 μl of 0.5M EDTA, and 2 mg chondroitin sulfate A is added to the reaction as carrier. The sample is applied to a small column (0.5 ml) of DEAE-Sephacel prepared in a disposable pipette tip and washed 3 times with 10 ml of 20 mM sodium acetate, pH 6.0, containing 0.2M NaCl. The polysaccharides are eluted using the same buffer containing 1M NaCl.

N-deacetylation and N-sulfation reactions are tightly coupled in vivo (free glucosamine residues are rarely found in heparan sulfate and heparin), even though each activity can be studied separately in vitro. The process of N-sulfation is not random and N-sulfated residues tend to be clustered and thus lead to domain organization as highly sulfated and non-sulfated regions along the polymer. There are four known N-deacetylase-N-sulfotransferases in humans (NDST1, NDST-2, NDST3, and NDST4), which are differentially expressed and regulated in different tissues. The ratio of N-deacetylase to N-sulfotransferase activities differs dramatically among the four isoforms.

Partial digestion of the N-deacetylase-N-sulfotransferased polysaccharide may then be carried out using heparinitases and analysed by LC/MS to confirm the identity of the product.

Enzymatic Epimerase Treatment of the N-Sulfated Polysaccharide: Step three of the polysaccharide biosynthesis is epimerase treatment of the N-sulfated polysaccharide using glucuronyl C-5 epimerase (Example 6). Glucuronyl C-5 epimerase catalyzes both the forward and reverse reaction of the inversion of the stereochemical configuration at the C-5 of one or more uronic acids along the chain, e.g., inter-conversion of glucuronic acid (GluA) to iduronic acid (IdoA). To date there are no efficient chemical strategies available to regio-, and stereo-, selectively epimerize GluA to IdoA. Epimerase treatment proceeds only when these residues are located at the reducing side of N-sulfated glucosamine residues, but it will not react with uronic acids that are O-sulfated or that are adjacent O-sulfated glucosamine residues. This suggests that epimerization occurs immediately after N-deacetylation and N-sulfation but before O-sulfation. This reaction results in the formation of N-sulfated heparan sulfate or heparin containing both iduronic and glucuronic acid residues (FIG. 3, Polysaccharide 3).

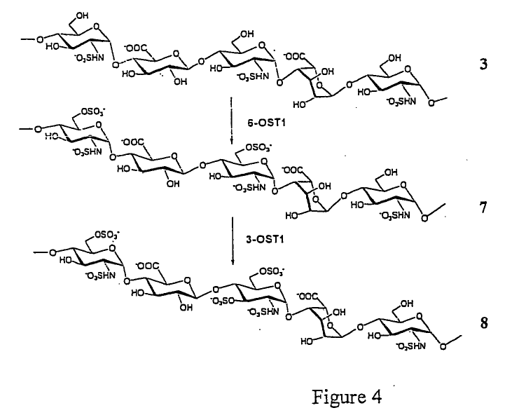

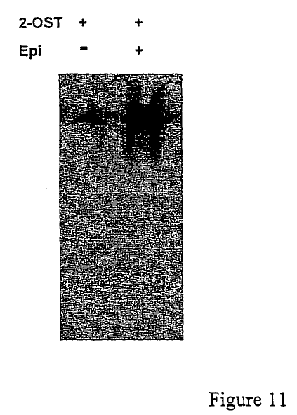

Epimerization is monitored by treating polysaccharide with radioactive [³⁵S]3′-phosphoadenosine 5′-phosphosulfate (PAP) in the presence of 2-OST1 and analyzed for the selective incorporation of radioactive sulfate into the polymer by 2-O sulfation of iduronic acid residues (FIG. 3, Polysaccharide 4) (Example 6). The ration of iduronic acid to glucuronic acid was about 85:15. Selective 2-O sulfation of iduronic acid on Polysaccharide 3 was observed, whereas incorporation of sulfate on Polysaccharide 2 was not observed (FIG. 11). In addition, non-epimerized, 2-O-sulfated polysaccharides may be obtained with the use of larger amounts of 2-OST1, more PAPS, and longer incubation times. In an embodiment, the N-sulfated polysaccharide (FIG. 4, Polysaccharide 3) was also treated with [³⁵S] PAP in the presence of 6-OST1 to prepare radioactive 6-O sulfated polysaccharide (FIG. 4, Polysaccharide 7) (similar to the Polysaccharide 5 illustrated in FIG. 3, but lacking 2-O sulfate groups) and detect the extent of epimerization and subjected to low pH nitrous acid and sodium borohydride treatment, which results in two disaccharides, GluA-anMan_{R} [6³⁵S] and IdoA-anMan_{R} [6³⁵S], that are resolved on C-18 reverse phase HPLC to determine the percentage of each epimer (Example 6).

Alternatively, the epimerization process is measured by incorporating ³H at C-5 using ³H₂O or by measuring the release of ³H from ³H-labeled (at C-5) N-sulfated polysaccharide (Li et al. (1997) J. Biol. Chem. 272(44):28158-63). In short, the reaction mixtures (total volume, 55 μl) contained 25 μl of cell lysate or medium, 25 μl of 2× epimerase assay buffer (20 mM HEPES, 30 mM EDTA, 0.02% Triton-X-100, and 200 mM KCl, pH 7.4), and 5 μl of substrate (10,000 cpm ³H). Alternatively, the reaction mixtures are applied to a PD-10 column (Pharmacia Biotech Inc.), and the released ³H in the ³H₂O form is separated from the polysaccharide substrate. The fractions containing ³H₂O form are evaporated by lyophilization. Enzymatic conversion of D-glucuronic acid to L-iduronic acid is demonstrated as follows. A sample (˜20 μg, 200,000 cpm ³H) of the modified polymer is incubated with 250 μl of cell lysate in a total volume of 300 μl of epimerase assay buffer at 37° C. for 6 hours. The incubation is terminated by heating at 100° C. for 5 min. The sample is mixed with 50 μg of carrier heparin and reacted with nitrous acid at pH 1-5, followed by reduction of the products with NaBH₄. The resultant hexuronylanhydromannitol disaccharides were recovered by gel chromatography on a column (1×200 cm) of Sephadex G-15 in 0.2 M NH₄HCO₃, lyophilized, and subjected to paper chromatography on Whatman No. 3 MM paper in ethyl acetate/acetic acid/water (3:1:1). Since abstraction of protein is a rate limiting step and ³H should have a primary isotopic effect in this process, quantification of the epimerization process is not considered efficient.

To date, there are no efficient chemical strategies available to selectively epimerase treat glucuronic acid to iduronic acid, or vice versa. In general, chemically synthesized iduronic acid containing a glycosyl donor or acceptor is prepared through several steps and attached as a single monosaccharide unit into the fully protected sugar. The presence of both epimers along the polysaccharide adds further complexity and challenges to chemical synthesis.

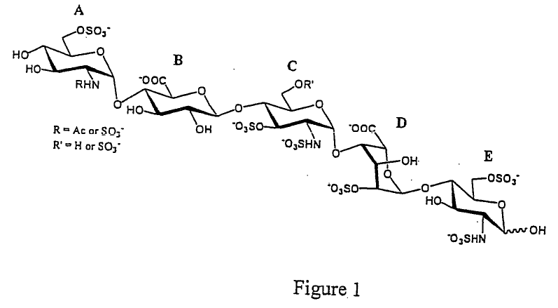

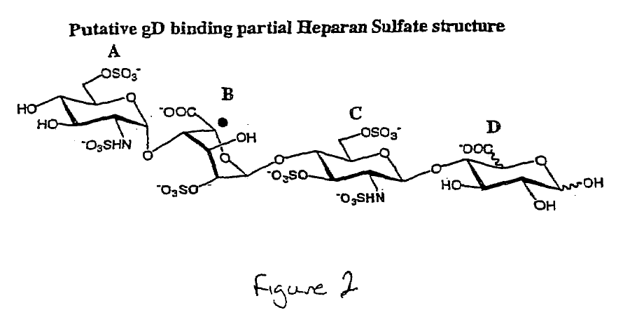

Enzymatic 2-O Sulfation of the N-Sulfated Polysaccharide: Step four of polysaccharide synthesis involves 2-O sulfation of the N-sulfated and epimerized polysaccharide using 2-OST1 to form an N-sulfated, 2-O sulfated polysaccharide (Example 7). It has been suggested that 2-O sulfation of iduronic acid within the AT-III binding pentasaccharide (Unit D of FIG. 1) limits 3-OST-1 mediated 3-O sulfation of heparan sulfate at the reducing end of IdoA (2S) residues, while it has no effect at its non-reducing end. Polysaccharide 4 of FIG. 3 was prepared from Polysaccharide 3 in the presence of PAPS or radioactive [³⁵S] PAP, catalyzed by 2-OST. The radioactive Polysaccharide 4 (FIG. 3) was analyzed by 5% native polyacrylamide gel to confirm the action of 2-OST.

Enzymatic 6-O Sulfation of the N-Sulfated, 2-O Sulfated Polysaccharide: Step five of polysaccharide synthesis involves 6-O sulfation of the N-sulfated, 2-O sulfated polysaccharide using 6-OST1 to form an N-sulfated, 2-O sulfated, 6-O sulfated Polysaccharide 5. Polysaccharide 5 of FIG. 3 was prepared from Polysaccharide 4 in the presence of PAPS or radioactive [³⁵S] PAP, catalyzed by 6-OST (Example 7). The 6-O labeled polysaccharides were digested to disaccharides by heparitinases (Example 7). Disaccharide analysis by C-18 HPLC confirmed the incorporation of 6-O sulfate groups as they eluted at the expected position along with standards (Example 7).

There are at least three main heparan sulfate 6-OST sulfotransferase isoforms that can be used in the methods of the invention: 6-OST1, 6-OST2, and 6-OST3; and there are two splice variants of 6-OST2, 6-OST2a and 6-OST2b. All of these isoforms have been cloned and expressed (Habuchi et al. (2000) J. Biol. Chem. 275: 2859-68). 6-OST1 preferentially sulfates IdoA-(or IdoA2S)-GlcNS. 6-OST2 preferentially sulfates GlcA-GlcNS residues. All three isoforms sulfate completely desulfated N-resulfated (CDSNS) heparin equally well. However, N sulfated heparosan was preferentially sulfated by these isoforms in the following order: 6-OST2>6-OST3>6-OST1 (Habuchi et al. (2000)). However, the results produced from the current in vitro studies suggest that these isoforms do not have significant differences in their substrate preferences for polysaccharides shown in FIG. 1. 6-OST is required for in vitro synthesis of ATIII binding polysaccharides. This is not the case for other isoforms. However, these latter isoforms may be necessary to produce polysaccharides that have biological functions other than anti-coagulation.

Enzymatic 3-O Sulfation of the N-Sulfated, 2-O Sulfated, 6-O Sulfated Polysaccharide: Step six of polysaccharide synthesis involves 3-O sulfation of the N-sulfated, 2-O sulfated, 6-O sulfated polysaccharide (FIG. 3, Polysaccharide 5) using 3-OST1 to form an N-sulfated, 2-O sulfated, 6-O sulfated, 3-O sulfated polysaccharide (FIG. 3, Polysaccharide 6) (Example 7). Polysaccharide 6 was prepared using radioactive [³⁵S] PAP or [³⁴S] PAP in the presence of 3-OST1, analyzed by 5% native polyacrylamide gel to confirm the action of 3-OST1, and purified using a mini DEAE column. The purified Polysaccharide 6 was digested with heparitinases and analyzed by C18 HPLC. The radioactive peaks, corresponding to 3-O sulfated disaccharide, and a resistant tetrasaccharide containing radioactive 3-O sulfate group, characteristic of 3-O-containing heparan sulfate polysaccharide, was eluted at the expected positions, demonstrating the incorporation of 3-O sulfate. Mass spectrometric analysis revealed the presence of stable isotope containing 3-O sulfated disaccharide and tetrasaccharide species, demonstrated the generation of Polysaccharide 6. The number of 3-O sulfates incorporated was 2-3 per 100 disaccharide residues, calculated from the ratio of radioactive 6-O sulfate and 3-O sulfate incorporation.

There are six known isoforms of heparan sulfate 3-O sulfotransferase, namely 3-OST1, 3-OST2, 3-OST3 (3-OST3a, 3-OST3b), 3-OST4, 3-OST5 and 3-OST6. 3-OST1 is primarily involved in generating anticoagulant heparan sulfate polysaccharide and generally acts on glucosamine residues flanked by the reducing side of GlcUA and non-reducing side of IdoA to generate AT III binding structures containing GlcUA-GlcNS3S and GlcUA-GlcNS3S6S. 3-O sulfate groups could not be incorporated at GlcN residues of Polysaccharide 2 using 3-OST1, suggesting a requirement of iduronic acid adjacent GlcN to be 3-O-sulfated. 3-OST3a preferentially produces gD binding heparan sulfate polysaccharide, enabling cellular entry of herpes simplex virus I (HSV). 3-OST5 generates both ATIII-binding heparan sulfate as well as gD-binding heparan sulfate. The functional significance of 3-OST2 and 3-OST4-modified heparan sulfate is still unknown. However, it is important to note that these isoforms are exclusively expressed in human brain, suggesting a role in neurobiology.

### Synthesis of a Modified Antithrombin III-Binding Heparan Sulfate Lacking 2-O Sulfation that Does Not Bind to Platelet Factor 4

Platelet Factor 4 (PF4) is a small polypeptide present in the α a granules of platelets.

When platelets are activated by an agonist, they release platelet factor 4, which binds to endothelial cell heparan sulfate and form a complex on the surface of endothelial cells. In aberrant circumstances, e.g., thrombosis, the complex serves as an antigen for the human immune system, resulting in the generation of antibodies that recognize the platelet factor 4-heparan sulfate complex. Antibody-antigen interaction activates the complement cascade, disrupting cell membranes and ultimately the control mechanisms that suppress the coagulation cascade. Damage to the blood vessel surface results in further activation of blood coagulation mechanism, local thrombosis, and decreased platelet levels. Similarly, administration of heparin causes a similar reaction, called heparin-induced thrombocytopenia (HIT), a very severe side effect of heparin therapy that can cause death. Inhibition of platelet factor 4-heparin binding would therefore help avoid these conditions.

Inhibition of platelet factor 4-heparin binding is also useful for enhancing the effectiveness of heparin therapy in certain patients. Heparin is not fully effective in platelet-rich regions of the body, probably because platelet factor 4 is released from platelet rich thrombi, which results in the on-going neutralization of heparin thus providing a sanctuary for activated products of the blood coagulation cascade. This series of events limits the effectiveness of heparin in the treatment of thrombosis in certain patients. Characterization of the binding site for protein factor 4 on heparin provides the information needed to redesign the binding site on heparin.

The key residues on the heparin molecule required for binding to platelet factor 4 appear to be the 2-O sulfated iduronic groups. By redesigning heparin to lack 2-O sulfated iduronic groups, the binding site on heparin for platelet factor 4 is not available. This provides several beneficial effects. The polysaccharide devoid of Ido(2S) residues acts as an anticoagulant while reducing the risk of fatality due to HIT. On the other hand, this non-classical anticoagulant should be more effective in areas with platelet enriched thrombi that release PF4 and generate a protected sanctuary for blood clotting. Heparin that is devoid of 2-O sulfated iduronic acid has fewer side effects and would be resistant to hepararnase cleavage, requiring the administration of a lower dosage to treat thrombotic lesions.

The 2-O sulfate of IdoA(2S) (FIG. 1, Unit D) is a minor contributing factor for AT III binding and hence its anticoagulant action. In addition, it was shown earlier that 3-OST1 can also act in the absence of 2-O sulfation and still generate the ATIII binding motif. One of the functions of 2-OST1 may be to restrict the action of 3-OST1 at certain glucosamine units located to the reducing side of IdoA(2S) along the chain.

Given that 2-O sulfate is not essential for anticoagulant action of heparan sulfate, a parallel synthetic strategy was developed to prepare anticoagulant heparan sulfate polysaccharides without 2-O sulfate-containing IdoU (adoA(2 S)) residues and to characterize the anticoagulant activity of the non-2-O sulfated heparan sulfate in terms of its ability to inhibit factor Xa (Example 10).

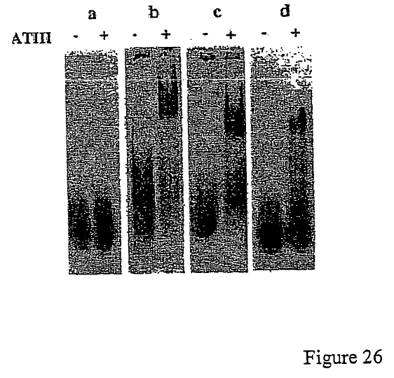

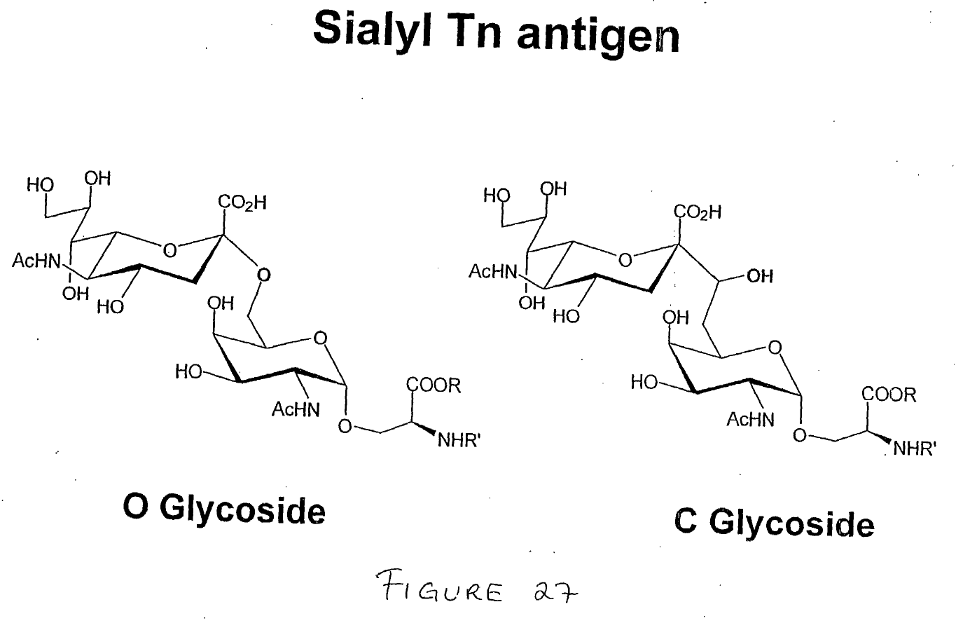

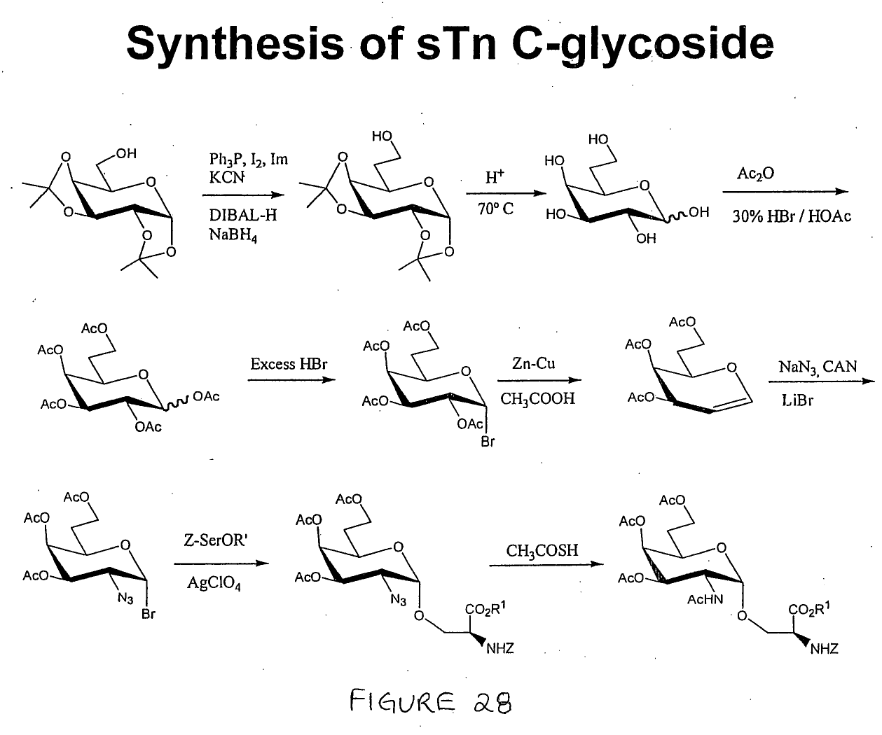

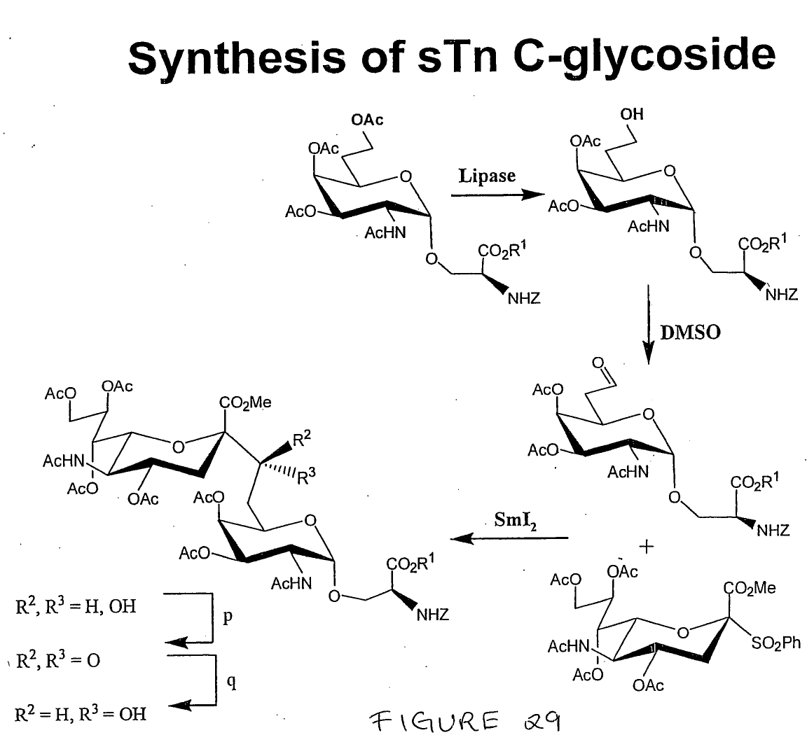

Polysaccharide 8 of FIG. 4 was prepared from Polysaccharide 3 in the presence of radioactive [³⁵S] PAP (Example 7). The N-sulfated heparan sulfate (FIG. 4, Polysaccharide 3) was treated with 6-OST1 to form N-sulfated, 6-O sulfated heparan sulfate (FIG. 4, Polysaccharide 7). The N-sulfated, 6-O sulfated heparan sulfate was then treated with 3-OST1 to form N-sulfated, 6-O sulfated, 3-O sulfated heparan sulfate (FIG. 4, Polysaccharide 8). Radioactive PAP was used to estimate sulfate incorporation. Gel mobility shift assay (GMSA) was carried out to determine ability of Polysaccharide 6 and Polysaccharide 8 to bind to AT III. Polysaccharide 5, lacking critical 3-O sulfate residue essential for binding to ATIII and anticoagulant activity, failed to bind to ATIII and hence not shifted (FIG. 26a) whereas Polysaccharide 6 and Polysaccharide 8 both bound to ATIII (FIGS. 26b and 26c). The resulting polysaccharide, which is devoid of 2-O sulfated iduronic acid, is still able to bind to antithrombin III and has fewer side effects due to its inability to bind to platelet factor 4. Since 2-O sulfate of Ido2S is a minor contributing factor for antithrombin III binding, the anticoagulation action of Polysaccharide 8 was not significantly affected. 2-O sulfation of iduronic acid within the antithrombin III-binding heparan sulfate limits 3-OST-1 mediated 3-O sulfation of heparan sulfate down stream of the Ido2S residue at its reducing end, while it has no effect at its non-reducing end. One of the functions of 2-OST1 may be to restrict the action of 3-OST1 at certain glucosamine units located to the reducing side of IdoUA(2S) along the polysaccharide.

### Synthesis of a Glycoprotein D (D)-Binding Heparan Sulfate

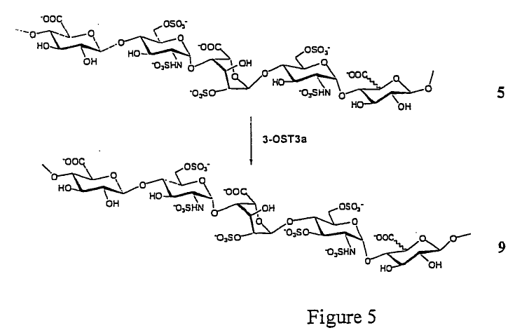

The methods of the invention were used to synthesize a heparan sulfate that specifically binds to glycoprotein D of herpes simplex virus 1 (HSV1). In this method, the isoform 3-OST3a was used to 3-O sulfated the N-sulfated, 2-O sulfated, 6-O sulfated polysaccharide (FIG. 5, Polysaccharide 5) to form an N-sulfated, 2-O sulfated, 6-O sulfated, 6-O sulfated polysaccharide (FIG. 5, Polysaccharide 9), as described herein (Example 7).

### Synthesis of Pentasaccharide 15

Oligosaccharides bearing biological activity are more desirable drugs and easier to use as probes to search for their protein ligands or to study genetic abnormalities. However, the data in the literature on the action of various HS biosynthetic enzymes has suggested that in many cases large oligosaccharides or polymers may be required for the action of biosynthetic enzymes such as C-5 epimerase. On the contrary, the invention provides compositions produced by methods of enzymatically synthesizing short polysaccharides or oligosaccharides of any size and any structure.

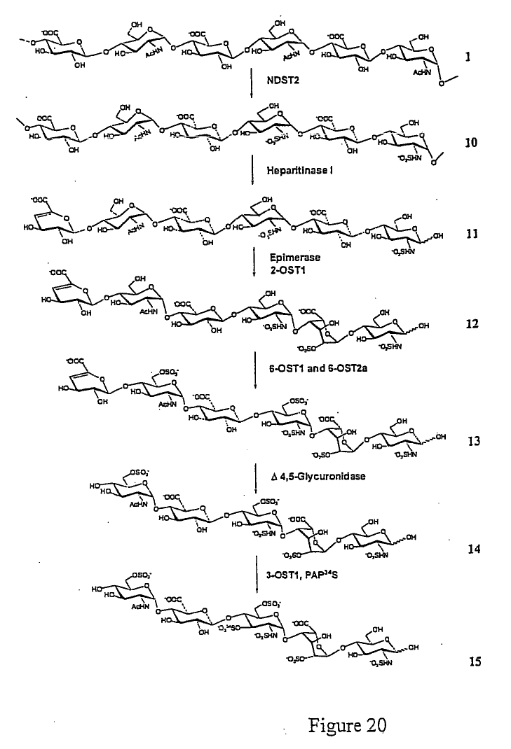

Referring to FIG. 20, Polysaccharide 1 was treated with NDST2 in the presence of PAPS to synthesize Polysaccharide 10, which contains both N-sulfated glucosamine and unmodified intact N-acetyl glucosamine residues. A small portion of the reaction mixture was withdrawn at regular intervals and extensively digested with heparitinase I to obtain disaccharides that were then analyzed by LC/MS to determine the extent of modification of Polysaccharide 1 by NDST2. The reaction was quenched when a desirable extent of modification, such as 70%, although other preferred percentages include any percent greater than 0%, such as about 10%, about 20%, about 30%, about 40%, about 50%, about 60%, about 80%, about 90, and about 100% of N-acetyl glucosamine residues that were N-deacetylated and N-sulfated, was reached.

Next, partial cleavage of the Polysaccharide 10 was performed to synthesize hexasaccharide 11. Briefly, the Polysaccharide 10 was treated with heparitinase I and the resulting oligosaccharide mixture of different sizes and different composition was purified to homogeneity using preparative HPLC. The fractions were analyzed by ESI-MS and those that contain hexasaccharide 11 with a molecular weight of 1213 Da were subjected to further analysis.

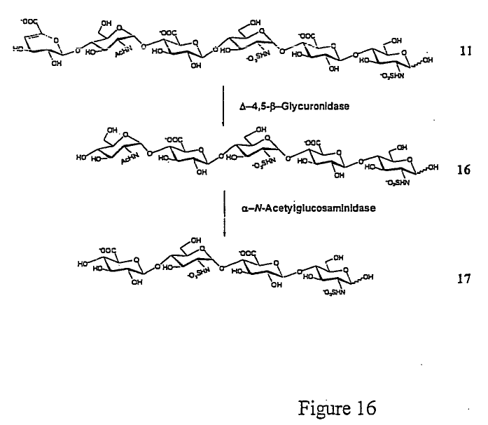

Referring now to FIG. 16, hexasaccharide 11 was treated sequentially with Δ^{4,5}glycuronidase and α-N-acetyl glucosaminidase, which resulted in Tetrasaccharide 17 with a molecular weight of 852 Da and thereby its structural identity was confirmed. If the penultimate residue of pentasaccharide 16 were N-sulfoglucosamine, such a residue would be resistant to α-N-acetyl glucosaminidase treatment and would have failed to yield the tetrasaccharide 17.

Referring again to FIG. 20, hexasaccharide 11 was then treated with C-5 epimerase and 2-O sulfotransferase 1 (2-OST1) to synthesize hexasaccharide 12. The C-5 epimerase can act only on the glucuronic acid flanked by N-sulfoglucosamine units and convert it to iduronic acid, whereas the glucuronic acid located at the reducing side of N-acetyl glucosamine can not be epimerized and thus treatment of hexasaccharide 12 with epimerase exclusively leads to the formation of a single product containing epimerized glucuronic acid next to glucosamine unit located at reducing end. 2-OST1 preferentially sulfates iduronic acid and rarely sulfates glucuronic acid located at the reducing side of N-sulfoglucosamine. Thus immediately after the action of epimerase upon hexasaccharide 11, preferential sulfation of the newly generated IdoA by 2-OST1 resulted in the formation of hexasaccharide 12. This tandem modification also prevents the reverse reaction catalyzed by epimerase. Given the fact that oligosaccharides are poor substrates for epimerase and that its action is reversible, the efficiency of this tandem modification, which is about 10-15%, is significant.

Referring still to FIG. 20, the hexasaccharide 12 was then treated with 6-O sulfotransferase 1 and 6-O sulfotransferase 2a to synthesize hexasaccharide 13. There are three 6-O sulfation sites available for 6-O sulfotransferases. The sites of 6-O sulfation were determined from the disaccharide analysis. Only two glucosamine residues located at the non-reducing end and middle of the hexasaccharide 13 were 6-O sulfated and the glucosamine unit at the reducing end was not modified. Hexasaccharide 13 was treated with Δ^{4,5}gycuronidase to remove the terminal unsaturated uronic residue at the non-reducing end which was generated by the action of heparitinases on Polysaccharide 10 and resulted in Pentasaccharide 14.

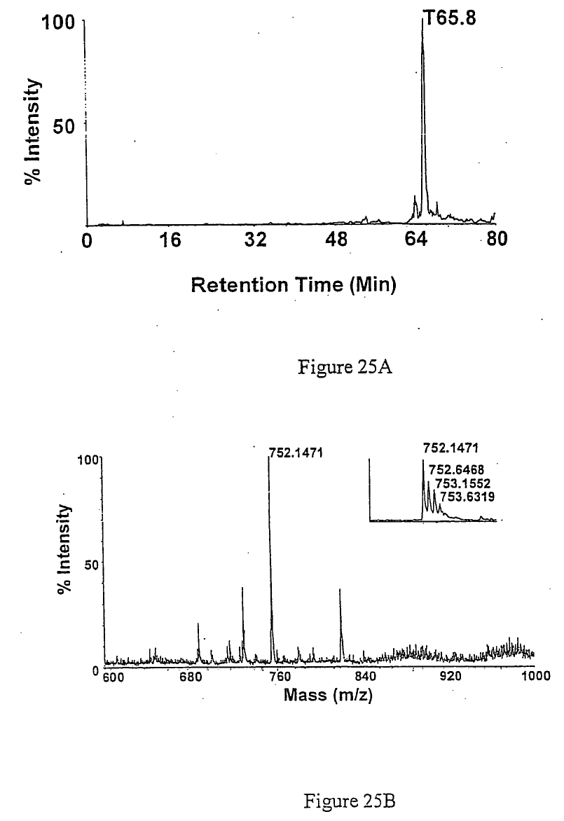

Referring still to FIG. 20, the final step was 3-O sulfation of Pentasaccharide 14 by 3-OST1 to generate anticoagulant Pentasaccharide 15. The final modification was carried out using PAPS enriched with ³⁴S isotope, which would serve as a mass spectral probe for structural characterization or radioactive PAP³⁵S for gel mobility shift analysis. The identity of the final product was verified by LC/MS which can distinguish pentasaccharides that differ both in number and placement of sulfate groups. The observed abundant molecular ion for Pentasaccharide 15 was 752.16 corresponding to [M-3H+DBA]⁻² which was consistent with the calculated molecular weight (FIG. 25B) GMSA confirmed that the Pentasaccharide 15 can bind to ATIII (FIG. 26d). Thus enzymatic synthesis of ATIII binding pentasaccharide was accomplished in 6 steps, a far fewer steps than chemical approaches requiring as many as 60 steps. The factor Xa assay confirmed their ability to bind to and accelerate the action of ATIII.

The invention provides methods for determining the critical or functional groups associated with a polysaccharide and altering those critical groups to change the binding characteristics of the polysaccharide. In an embodiment, the critical groups on a subject's polysaccharides may not be completely modified (e.g., sulfated), thereby altering the polysaccharide's ability to bind to a binding partner. The polysaccharide may then be modified in vitro to determine whether the binding to the binding partner has been restored. The polysaccharides may be modified, e.g., sulfated, epimerized, etc., to contain critical groups thereon. The number and/or combination of critical groups may represent or the in vivo state of modification of the polysaccharide. Alternatively, the number and/or combination of critical groups may be customized to study the effect of the presence or absence of additional, fewer, or rearranged critical groups on the polysaccharide or the effect on the biosynthetic pathway of the modified or unmodified polysaccharide. In an embodiment, the number and/or combination of critical groups may be incomplete or partial, such that the number and/or combination of critical groups mimics the natural state of a polysaccharide at a certain point in the in vivo modification pathway, or in order to represent the number and/or combination of critical groups that are present on a polysaccharide under certain conditions, e.g., at different stages of development, in a disease state, etc.

The practice of the present invention can employ, unless otherwise indicated, conventional techniques of protein and polysaccharide chemistry, enzymologist, cell biology, cell culture, molecular biology, microbiology, and recombinant DNA, which are within the skill of the art. Such techniques are explained fully in the literature.

Practice of the invention will be still more fully understood from the following examples, which are presented herein for illustration only and should not be construed as limiting the invention in any way. Variations and alternate embodiments will be apparent to those of skill in the art. The contents of all cited references (including literature references, issued patents, and published patent applications that may be cited throughout this application) are hereby expressly incorporated by reference.

### EXEMPLIFICATION

### Example 1

### Materials

Heparan sulfate K5 precursor polysaccharide was prepared from E. coli K5 strain (Example 2). A human C5 epimerase cDNA clone was isolated from a human fetal brain cDNA library (Example 3), and expressed in baculovirus (Example 4). Heparan sulfate sulfotransferases, 2-OST1, 3-OST1, 3-OST3a, 6-OST, and C-5 epimerase were all cloned and expressed in a baculovirus system and purified as described in Liu et al. (1999) J. Biol. Chem. 274:5185-92. [³⁵S] PAPS and [³⁴S] PAPS were prepared as reported previously (Wu et al. (2002) FASEB J. 16:539-45) whereas [³²S] PAPS was purchased from Calbiochem. Heparitinase I (EC 4.2.2.8), heparitinase II, and heparinase (EC 4.2.2.7) were obtained from Seikagagu America, Falmouth, Mass. Factor Xa was obtained from Haematologic Technologies, Essex Junction, Vt. Antithrombin III was obtained from GlycoMed, San Diego, Calif. All chemicals were purchased from Sigma unless otherwise indicated. Δ^{4,5}Glycuronidase (no EC number) was from Seikagagu Corp., Tokyo, Japan. α-N-acetylglucosaminidase (3.2.1.50) was from Glyko, Calif., USA.

### Example 2

### Preparation of Heparan Sulfate K5 Precursor Polysaccharide from E. coli K5 Strain

Heparan sulfate K5 precursor polysaccharide was prepared from E. coli K5 strain as described in Vann et al. (1981) Eur. J. Biochem. 116:359-64. Briefly, the acidic capsular polysaccharide and bacterial cells were precipitated from liquid cultures by the addition of an equal volume of 0.2% hexadecyltrimethylammonium bromide (Cetavlon). The polysaccharide was extracted from the precipitate with 1 M calcium chloride. The polysaccharide was purified by three cycles of precipitation with ethanol (80%) followed by extraction with phenol, buffered with sodium acetate to pH 6.5. The final aqueous phase was ultracentrifuged for 2 hours at 105,000× g and the supernatant was freeze-dried. All operations were carried out at 4° C. Mass spectroscopy analysis of smaller fragments was in accordance with the calculated molecular weight and thus confirms the identity of heparan sulfate precursor polysaccharide structure isolated from bacterial culture.

### Example 2a

### Production of K5 Polysaccharide

In a preferred method, heparan sulfate K5 precursor polysaccharide was also prepared as follows. E. coli K5 bacterial cells were grown overnight in 1 L of growth medium containing following ingredients: casaminoacids (20 g), yeast extract (10 g), NaH₂PO₄ (4.8 g), KH₂PO₄ (4.2 g), K₂HPO₄ (5.3 g), MgCl₂ (0.5 g), glucose (2 g). FeSO₄ (20 mg). The pH of the bacterial culture was adjusted to 6.0 with acetic acid, solid protease (200 mg/L) was added and kept at 37° C. for 24 hours. Insoluble material was removed by centrifugation at 3000 rpm. The supernatant was diluted with an equal volume of double distilled water and applied to a DEAE-Sephacel column (50 ml) that was previously equilibrated with washing buffer (0.2 M NaCl in 20 mM sodium acetate, pH 6). The column was washed with 20 bed volumes of washing buffer and the K5 polysaccharide was eluted with 0.5 M NaCl in 20 mM sodium acetate containing 0.01% TRX-100 (pH 6). The eluate was adjusted to 1M NaCl with solid sodium chloride, and then 4 volumes of cold ethanol was added and left overnight at 4° C. to precipitate N-acetyl heparosan. The precipitate was centrifuged at 3000 rpm for 30 minutes and subsequently vacuum dried. The isolated Polysaccharide 1 was digested with heparitinases and analyzed by LC/MS. The m/z of 378 [M-H]⁻¹ and 757 [M-H]⁻¹ were observed for disaccharide and tetrasaccharide molecular ions.

### Example 3

### Cloning and Expression of Human Glucuronyl C5 Epimerase

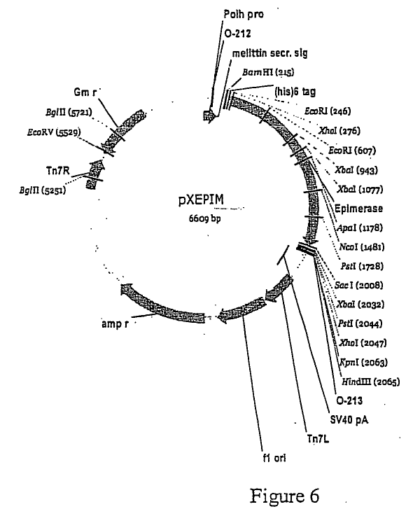

A cDNA clone encoding human C5 epimerase was isolated from a human fetal brain cDNA panel (Origene, Rockville, Md.) by screening with PCR primers that amplified a segment spanning nucleotides 7-157 of the coding region of the human C5 epimerase gene (Genbank number XM 035390), using standard techniques. A donor plasmid for the preparation of recombinant baculovirus expressing a soluble form of the human C5 epimerase was constructed in a pFastBac HT plasmid (Gibco, Grand Island, N.Y.) modified by the insertion of the honeybee melittin signal peptide 5′ of a histidine tag as described in Liu et al. (1999) J. Biol. Chem. 274: 5185-92. The construction of the modified donor plasmid, pXEPIM, employed a synthetic oligonucleotide adapter that also encoded amino acids 35-44 of human C5 epimerase and two restriction fragments isolated from the cDNA clone (TaqI to EcoRI and EcoRI to SacI) that incorporate the rest of the epimerase coding region (FIG. 6).

### Example 4

### Baculovirus Expression and Purification of Glucuronyl C5 Epimerase

Human glucuronyl C5 epimerase recombinant baculovirus was prepared using the donor and the Bac-to-Bac baculovirus expression system (Life Technologies, Inc., Grand Island, N.Y.) according to the manufacturer's protocol, except that recombinant bacmid DNA was purified using an endotoxin-free plasmid purification kit (Qiagen, Inc., Valencia, Calif.) and transfection of Sf9 cells was scaled up to employ 15 μg of bacmid DNA and 2.5×10⁷ exponentially growing cells in four 100-mm dishes. Medium containing recombinant baculovirus was harvested at 3 days post-transfection and amplified twice for about 65 hours each on Sf9 cells. The resulting high-titer viral stock was stored in 0.75 ml aliquots sufficient to infect 3.5×10⁸ cells, as determined by Western blotting of medium from infected cells using (his)₄ antibody (Qiagen) according to standard methods. Infected cells were plated in ten 150 mm dishes and incubated at 26° C. for 90-96 hours. The pooled medium was centrifuged at 400×g, adjusted to 10 mM in HEPES, titrated to pH 7.4, chilled on ice for 30 minutes and centrifuged at 16,000×g. The clarified pool was diluted 1:1 with 10 mM HEPES, pH 7.4, made 1 mM in PMSF, and applied to an 8 ml column of ToyoPearl AF heparin 650M (TOSOHAAS, Montgomeryville, Pa.). The column was washed with 40 ml of HCG 50 (10 mM HEPES, pH 7.4, 2% glycerol, 0.6% CHAPS, 50 mM NaCl) and eluted with an 80 ml linear gradient of 50 to 600 mM NaCl in HCG. Aliquots of selected 1 ml fractions were analyzed by western blotting for the presence of the histidine tag, adjusted to 500 mM in NaCl, 10 mM in imidazole and concentrated using a YM-10 membrane (Amicon, Bedford Mass.) to about 3 ml.

### Example 5

### N-Deacetylation/N-sulfation of K5 Polysaccharide

30 mg of K5 polysaccharide was dissolved in 7.5 ml of 2N NaOH, incubated for 24 hours at 60° C., cooled to room temperature, and the pH adjusted to pH 7. The solution was warmed to 45-50° C. and 100 mg of sodium carbonate and 100 mg of trimethylamine-sulfur trioxide complex were added simultaneously and incubated for 12 hours. Another 100 mg each of sodium carbonate and trimethylamine-sulfur trioxide were added after 12 hours and the selective N-sulfation continued for additional 12 hours at 45-50° C. The solution was then brought to room temperature, dialyzed against distilled water overnight using a 1000 Dalton molecular weight cut-off cellulose membrane (Spectrum Laboratories, Rancho Dominguez, Calif.) and the dialysate was lyophilized to obtain salt-free N-sulfated K5 heparosan polysaccharide. The purified Polysaccharide 2 was digested with heparitinases and the resulting mixture was analyzed by LC/MS. The m/z of 416 was observed for the disaccharide corresponding to [M-H]⁻¹.

### Example 6

### Glucuronyl C5 Epimerase Reaction

N-sulfated K5 heparosan polysaccharide (1 μg) was incubated with purified 10-20 ng of glucuronyl C5 epimerase (Example 3) at 37° C. in a volume of 50 μl containing 25 mM HEPES, 40 mM CaCl₂, pH 6.5, or alternatively in 25 mM MES (pH 7.0) with or without polymer p40. After incubation for 12 hours, the same amount of C-5 epimerase was added again and the incubation was extended for an additional 24 hours (U.S. Pat. No. 5,958,899). The reaction mixture was diluted to 1 ml with DEAE wash buffer (0.25M NaCl, 20 nM Sodium Acetate, 0.01% Triton X pH 6.0) and purified on a 0.1 ml DEAE column (Pharmacia, Uppsala, Sweden). The epimerase-treated N-sulfated K5 polysaccharide was treated with PAP[³⁵S] in the presence of 10-30 ng 2-OST sulfotransferase in a total volume of 10 μl for 30 minutes (Wu et al. (2002) FASEB J. 16:539-45) and the reaction mixture was analyzed on 5% native polyacrylamide gel. The amount of enzyme used may be altered depending upon the incubation time, e.g., for incubations from 30 minutes to 2 days.

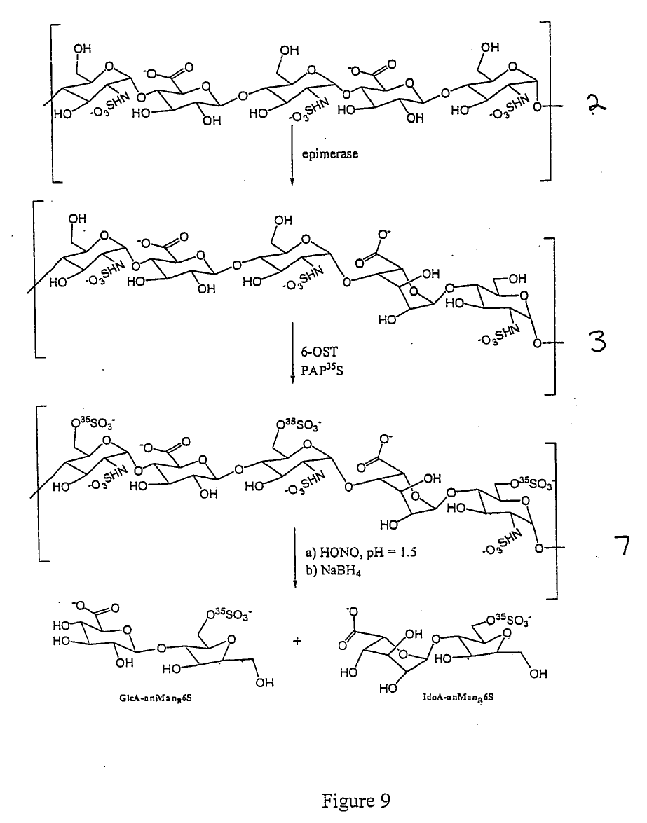

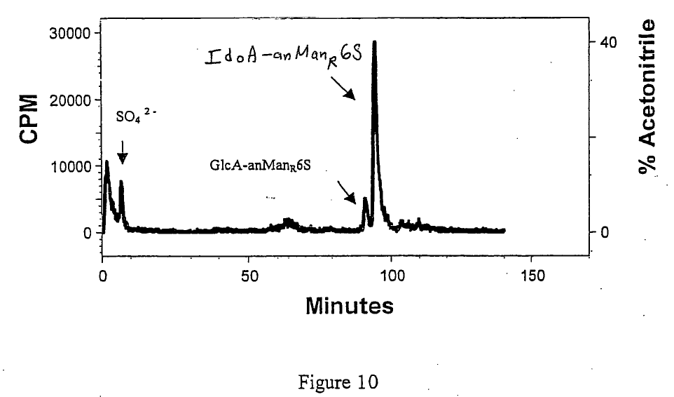

The epimerase-treated N-sulfated K5 polysaccharide was also treated with [³⁵S] PAP in the presence of 6-OST1 or 6-OST2a (Wu et al. (2002) FASEB J. 16:539-45). After DEAE purification, the polysaccharide was subjected to low pH nitrous and sodium borohydride treatment to obtain the disaccharide mixtures. The disaccharide profile was analyzed by HPLC on a C18 column (Vydac, Hesperia, Calif.) to estimate the percentage of glucuronic acid and iduronic acid (FIGS. 9 and 10).

### Example 7

### Selective Sulfation with Various Recombinant Sulfotransferases: NDST2, 2-OST1, 6-OST1, 6-OST2a, 6-OST2b, 3-OST1and 3-OST3a

NDST2 modified Polysaccharide 1 was used as the starting material for the following reactions. For selective sulfation of the epimerase treated N-sulfated K5 polysaccharide with various recombinant sulfotransferases, 1 μg of epimerase treated N-sulfated K5 polysaccharide was combined with 12.5 μl of 2× buffer (50 mM MES (pH 7.0), 1% (w/v) triton X-100, 5 mM MgCl₂, 5 mM MnCl₂, 2.5 mM CaCl₂, 0.075 mg/ml protamine chloride, 1.5 mg/ml BSA), 70 ng of the expressed sulfotransferase, 2-10 μl [³⁵S] PAPS, [³²S] PAPS, or [³⁴S] PAPS (˜1.0×10⁷ cpm), and the appropriate amount of water to 25 μl. The reaction was incubated at 37° C. for various times ranging from 30 minutes to overnight, diluted to 1 ml with DEAE wash buffer and purified on a DEAE column (Pharmacia). Alternatively, the reaction was stopped by heating at 70° C. and the reaction mixture was centrifuged at 10,000 g for 3 minutes and the supernatant was used for gel mobility shift assay (GMSA) (Example 8) or polyacrylamide gel analysis, according to studded methods. Sulfated polysaccharide each stage was digested with heparitinases I, II, and III and analyzed by ion pair reversed phase HPLC (IPRP-HPLC) (Vydac, Hesperia, Calif.). In the case of the 3-OST1 modification, [³⁴S] PAPS was used as a sulfate group donor and the digested polysaccharide was analyzed by high performance liquid chromatography coupled to micro-electrospray ionization time of flight mass spectroscopy (HPLC-ESI-TOF-MS) (Perceptive Biosystems, Framingham, Mass.). In the case of the 3OST1 modification, [³⁵S]PAPS or [³⁴S]PAPS was used as a donor and the digested polysaccharide was analyzed by macro HPLC or capillary HPLC-ESI-TOF-MS, respectively.

### Example 8

### Purification of Polysaccharides

After termination of sulfation, the reaction mixture was diluted to 1 mL with 0.25 M NaCl, 20 mM NaAc, 0.01% TX-100, pH 6.0 and 1 mg of glycogen was added to minimize nonspecific interaction of polysaccharides with the column matrix. The diluted reaction mixture was loaded onto a 0.1 mL DEAE-Sephacel column (Pharmacia, Uppsala, Sweden), pre-equilibrated with 2 mL of washing buffer containing 0.25 M NaCl, 20 mM NaAc, 0.01% TX-100, pH 6.0. The column was washed with 20 column volumes of washing buffer and the polysaccharide was eluted from the column with 2 mL of 1 M NaCl in 20 mM NaAc, pH 6.0. A 8 mL of absolute ethanol and 1 mg of glycogen were added to 2 mL of eluent in a 50 mL disposable polystyrene tube and incubated at 4° C. overnight to facilitate the precipitation of polysaccharide. The precipitate was obtained by centrifuging in an RC3B centrifuge for 15 minutes at 3000 rpm. The pellet was washed with 1 mL of 70% ethanol twice and finally dissolved in 200 μL of double distilled water for subsequent characterization.

### Example 9

### Gel Mobility Shift Assay

Radiolabeled polysaccharide (˜10,000 cpm) was mixed with 2 μg antithrombin III (AT-III) or glycoprotein D (gD) of herpes simplex virus I in binding buffer (12% glycerol, 20 mM Tris-HCl (pH 7.9), 100 mM KCl, 1 mM EDTA and 1 mM DTT in 20 μl. The reaction mixture was incubated at 23° C. for 20 minutes and was applied to a 4.5% native polyacrylamide gel (with 0.1% of bis-acrylamide). The gel buffer was 10 mM Tris (pH 7.4) and 1 mM EDTA, and the electrophoresis buffer was 40 mM Tris (pH 8.0), 40 mM acetic acid, 1 mM EDTA. The gel was run at 6 volts/cm for 1-2 hours with an SE 250 Mighty Small II gel apparatus (Hoefer Scientific Instruments, San Francisco). After electrophoresis, the gel was transferred to 3 MM paper and dried under vacuum. The dried gel was autoradiographed using a PhosphorImager 445SI (Molecular Dynamics, Sunnyvale, Calif.). The image was analyzed using NIH Image 1.60 and the band intensities were evaluated. The percentage of bound and free polysaccharide in the binding reaction was derived from the ratio of the respective bands.

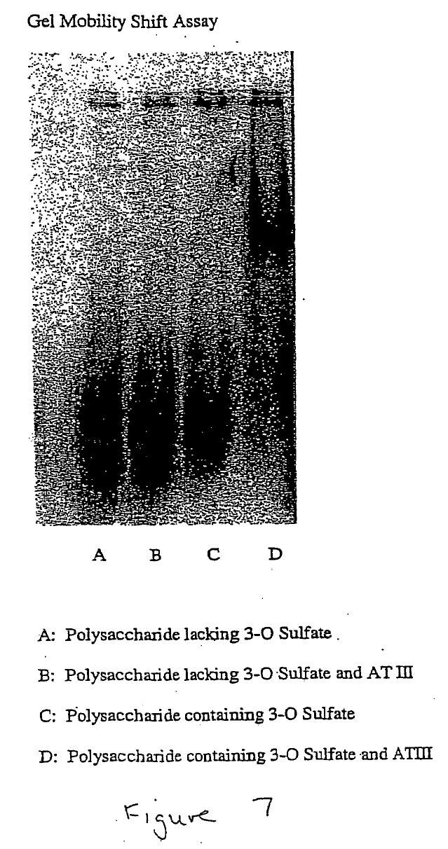

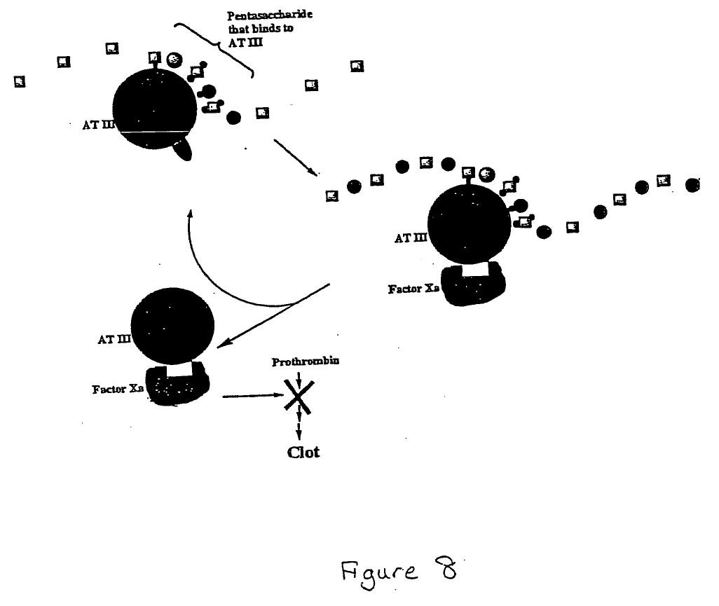

FIG. 7 illustrates the selective binding of heparan sulfate polysaccharide for AT-III that is 3-O sulfated and therefore only binds to and shifts AT-III.

### Example 10

### AT-III Inactivation of Factor Xa Assay

An AT-III Inactivation of Factor Xa assay was carried out to determine the anticoagulant activity of synthesized modified polysaccharides. Human factor Xa (10.4 mg/ml in 50% glycerol, 820 units/mg) was obtained from Hematologic Technologies, Essex Junction, Vt. Factor Xa (Haematologic Technologies) and antithrombin III (GlycoMed) were diluted 1:200 with PBS containing 1 mg of BSA (4 units/ml and 15 units/ml, respectively). AT-III (2.5 mg/ml) was diluted 1:200 to give a 2×10⁻⁷ M stock solution. The chromogenic substrate S-2765 was obtained from Chromogenix (West Chester, Ohio) and the stock solution of 1 mM with 1 mg/ml polybrene in water was prepared. Heparin (174 international units/mg, Sigma) was used as a standard.

Factor Xa was inactivated as follows. 25 μl of AT-III (2×10⁻⁷ M) was added to 25 μl of a serial dilution of heparin standards (Sigma, St. Louis, Mo.) or synthetic polysaccharide in Tris-EDTA buffer (50 mM Tris, 7.5 mM EDTA, and 175 mM NaCl (pH 8.4)). The reaction was incubated at 37° C. for 75 seconds. Factor Xa (25 μl, 4 units/ml) was added to the reaction. After incubating at 37° C. for 195 seconds, 25 μl of S-2765 was added to the reaction. The absorbance at 405 nm of the reaction was read every minute for 10 minutes using a Beckman UV spectrometer. FIG. 8 illustrates the mechanism of action of AT-III binding to heparan sulfate polysaccharide.

### Example 11

### Digestion of 3-O-Sulfated Polysaccharides 6 and 8 with Heparitinase I, Heparitinase II, and Heparinase

Heparitinase I recognizes the following sequences: GlcNAc/NS±6S(3S?)-GlcUA-GlcNAc/NS±6S. Heparitinase II has broad sequence recognition, GlcNAc/NS±6S(3S?)-GlcUA/IdceA±2S-GlcNAc/NS±6S. Heparinase recognizes the sequences: GlcNAc/NS±3S±6S-IdceA2S-GlcNAc/NS±6S. 3-O-³⁵S-Sulfate labeled polysaccharides were digested with 1 mU of Hep 1, II, and/or III in a total volume of 100 μl of 40 mM ammonium acetate containing 1 mM calcium chloride buffer (pH 7.0) at 37° C. overnight. An equal volume of ion-pairing agent solution (368 mM of tetrabutylammonium dihydrogen phosphate) was added to the reaction mixture, which was then combined with six HS disaccharide UV standards (Seikagaku America, Falmouth, Mass.) in a total volume of 200 μl and analyzed by C18 HPLC. In the case of LC/MS analysis, dibutylammonium acetate was used as ion-pairing agent and the details of which are given below.

### Example 12

### Flow Injection Capillary Liquid Chromatography

An Ultimate capillary HPLC workstation (Dionex, Sunnyvale, Calif.) was used for microseparation. UltiChrom software (Dionex) was used in data acquisition and analysis. A gradient elution was performed, using a binary solvent system composed of water (eluent A) and 70% aqueous methanol (eluent B), both containing 8 mM acetic acid and 5 mM dibutylamine as an ion-pairing agent. HPLC separations were performed on a 0.3 mm×250 mm C18 polymeric silica column (Vydac)). The column temperature was maintained at 25° C. and the flow rate was set to 5 μL min⁻¹. Sample volumes of 10 μl were injected. For disaccharide analysis, a 5 μL sample injection loop was used. The chromatographic conditions were optimized for resolution of disaccharides. In brief, nonsulfated disaccharide was eluted with 100% A, single sulfated disaccharides were elated with 10% B, isocratic elution with 20% B was used to obtain double sulfated disaccharides, followed by isocratic elution with 35% B to obtain triple sulfated disaccharides. The column was washed and equilibrated by further elution with 100% B for 10 minutes, returning to 100% A for 10 minutes at the end of the run. The absorbance of the column eluate was monitored at 232 nm.

### Example 13

### Mass Spectrometry

Mass spectra were acquired on a Mariner BioSpectrometry Workstation ESI time-of-flight mass spectrometer (PerSeptive Biosystems, Framingham, Mass.). In the negative-ion mode, the instrument was calibrated with bis-trifluoromethyl benzoic acid, heptadecafluorononanoic acid, perfluorotetradecanoic acid, and oligothymidylic acids sodium salt [d(pT)₆ and d(pT)₁₀]. Nitrogen was used as a desolvation gas as well as a nebulizer. Conditions for ESI-MS were as follows: nebulizer flow 1 L/min, nozzle temperature 140° C., drying gas (N₂) flow 0.6 L/min, spray tip potential 2.8 kV, nozzle potential 70 V, and skimmer potential 9 V. Negative ion spectra were generated by scanning the range of m/z 40-2000. During analyses, the indicated vacuum was 2.1×10⁻⁶ Torr.

### Example 14

### High Performance Liquid Chromatography

Separation and characterization of ³⁵S-labeled disaccharides were carried out by HPLC using a C18-reversed phase column (0.46×25 cm) (RPIP-HPLC) (Vydac). Solvent A contained double distilled water and 40 mM ammonium dihydrophosphate and 1 mM tetrabutylammonium dihydrophosphate. Solvent B contained 50% acetonitrile, 40 mM ammonium dihydrophosphate and 1 mM tetrabutylammonium dihydrophosphate. The RPIP-HPLC was eluted at a flow rate of 0.5 ml/minutes with the following stepwise gradients: 5% solvent B for 15 minutes; 12% solvent B for 15 minutes; 21% solvent B for 45 minutes; 36% solvent B for 25 minutes; 100% solvent B for 20 minutes and finally followed by 5% solvent B for 20 minutes to equilibrate the column.

In the case of analysis of disaccharides resulting from nitrous acid/sodium borohydride reduction, the following condition was employed. Solvent A contained double distilled water, 10 mM ammonium dihydrophosphate, and 1 mM tetrabutylammonium dihydrophosphate. Solvent B contained 40% acetonitrile, 10 mM ammonium dihydrophosphate, and 1 mM tetrabutylammonium dihydrophosphate. The RPIP-HPLC was eluted at a flow rate of 0.5 ml/min with following stepwise gradients: 100% solvent A for 15 minutes; 6% solvent B for 25 minutes; 12% solvent B for 40 minutes; 40% solvent B for 60 minutes; 100% solvent B for 10 minutes and finally followed by 100% solvent A for 20 minutes to equilibrate the column.

### Example 15

### Nitrous Acid/Sodium Borohydride Treatment of Polysaccharide 7

Referring to FIG. 9, Polysaccharide 7 was treated with nitrous acid/sodium borohydride to yield GlcA-anMan_{R}6S and IdoA-anMan_{R}6S (Polysaccharide 12). Briefly, radiolabeled Polysaccharide 7 was dried with 20 μg of cold Heparan sulfate carrier in 20 μL. 20 μL of the low pH HONO solution (0.5 ml of 0.5 M barium nitrite solution added to 0.5 ml of 0.5 M sulfuric acid solution, the precipitate spun down and supernatant placed on ice) was added and the reaction incubated at room temperature for 10 minutes. 1 μL phenol red solution (1 mg/ml) was added to each sample and the color was adjusted to pink with sodium carbonate. 5 μL of 1 M sodium borohydride, 0.01 M NaOH was added and incubated at room temperature 15 minutes. Excess sodium borohydride was destroyed by adding 10 μL of 2N acetic acid and the pH adjusted to 7 with sodium carbonate. The HPLC profile of the GlcA-anMan_{R}6S and IdoA-anMan_{R}6S mixture is shown in FIG. 10.

### Example 16

### Synthesis of Polysaccharide 4

Synthesis of Polysaccharide 4 was observed on 5% polyacrylamide gel (FIG. 11). 2-O sulfation of Polysaccharide 2 failed in the absence of epimerase treatment in the presence of radioactive PAPS and 2-OST1 (FIG. 11, Lane 1), whereas 2-O sulfation of Polysaccharide 3 resulted in the synthesis of Polysaccharide 4 (FIG. 11, Lane 2).

Alternatively, a 2-O-sulfated glucuronic acid-containing polysaccharide may be produced if epimerization is omitted and the amounts of 2-OST and PAP in the reaction increased, together with an increase in reaction time.

### Example 17

### Synthesis of Polysaccharides 5 and 7

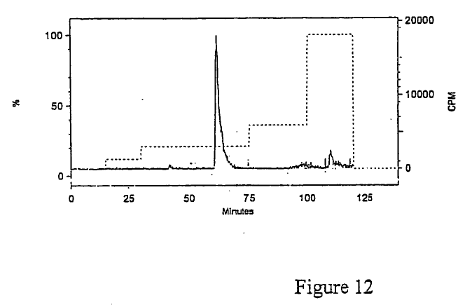

6-O sulfation of Polysaccharides 4 and 3 was performed to synthesize Polysaccharides 5 and 7, respectively (FIGS. 3 and 4). Polysaccharide 7 was synthesized by treating Polysaccharide 3 with radioactive PAPS and 6-OST (e.g., 6-OST1, 6-OST-2a, and 6-OST2b) as described in Example 7. The reaction product was purified on DEAE column, digested with heparitinases, and analyzed by C18 HPLC. Radioactive disulfated disaccharide (UA-GlcNS6³⁵S) migrated at the expected position along with the commercially available standard, cold disaccharide, which has a UV absorbance at 232 nm. The chromatogram is shown in FIG. 12.

### Example 18

### Synthesis of Polysaccharide 6 and 8

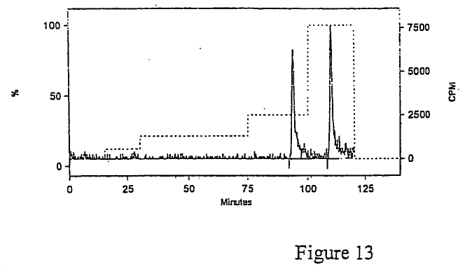

3-O-sulfation of Polysaccharides 5 and 7 was carried out in the presence of 3-OST1 and PAP³⁵S and the resultant Polysaccharides 6 and 8, respectively were treated with heparanitases. The digested Polysaccharides 6 and 8 were then analyzed by HPLC. FIG. 13 shows the HPLC profile of digested Polysaccharide 8. Both 3-O-sulfated disaccharide and 3-O-sulfated tetrasaccharide were present.

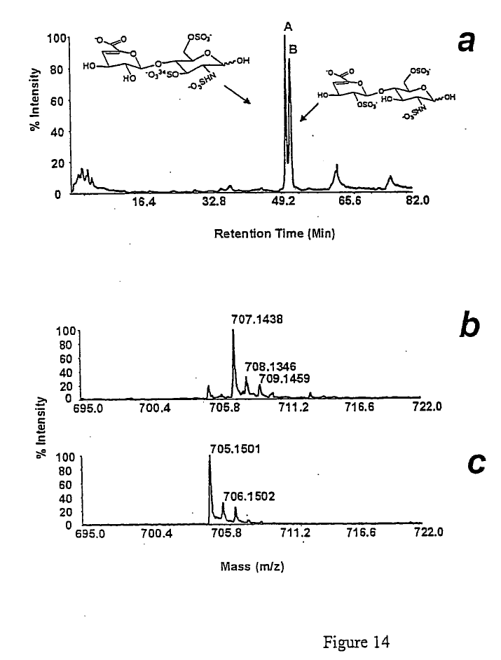

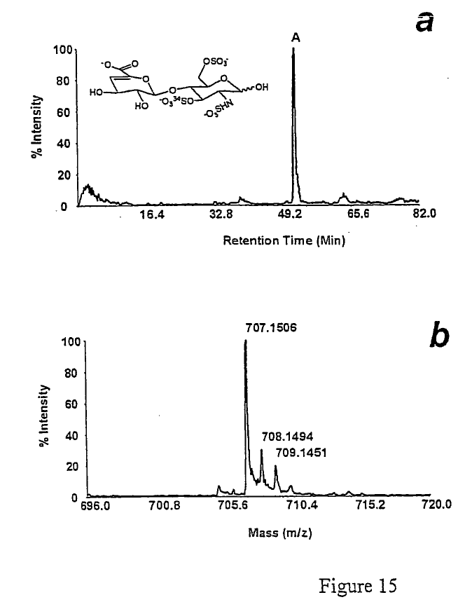

The 3-O-sulfation of Polysaccharides 5 and 7 also was carried out using PAP³⁴S and analyzed by LC/MS. The XIC of trisulfated disaccharide profile of Polysaccharide 6 is shown in FIG. 14A. The mass spectra of trisulfated disaccharides of Polysaccharide 6 is shown in FIG. 14B and FIG. 14C, which correspond to peaks A and B of FIG. 14A, respectively. The XIC of trisulfated disaccharide Polysaccharide 8 is shown in FIG. 15A. The mass spectra of Polysaccharide 8 is shown in FIG. 15B.

### Example 19

### Synthesis of Hexasaccharide 11, Pentasaccharide 16, and Tetrasaccharide 17

Polysaccharide 1 (50 μg) was resuspended in 100 μl of 2× sulfotransferase buffer. 90 μl Water, 10 μl PAPS (20 mg/ml) and 1 μl of NDST2 were added and N-sulfation was carried out at 37° C. Small aliquots were withdrawn for mass spectrometric analysis. The reaction was quenched when approximately 70% of glucosamine units were N-deacetylated and N-sulfated as indicated by LC/MS analysis. Partial digestion of Polysaccharide 10 (FIG. 20) with heparitinases was carried out and the resulting oligosaccharides were purified on a C18 column using HPLC. The individual fractions were analyzed by mass spectrometry and the fractions containing hexasaccharide 11 were pooled and dried.

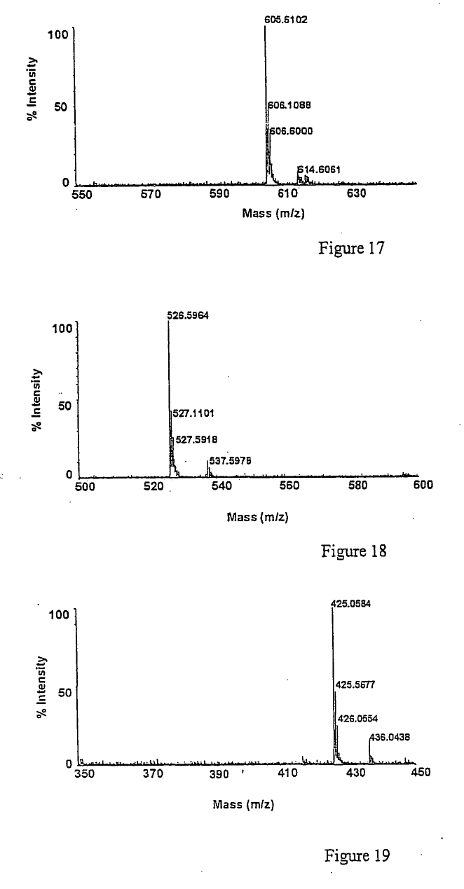

FIG. 16 illustrates the synthesis of Pentasaccharide 16 and Tetrasaccharide 17 from hexasaccharide 11. Hexasaccharide 11 (molecular weight is 1213 Daltons) was treated with Δ^{4,5}-glycuronidase to obtain Pentasaccharide 16 (molecular weight is 1055 Daltons), which was then treated with α-N-acetylglucosaminidase resulting in generation of Tetrasaccharide 17 (molecular weight is 852 Daltons). Successful generation of Tetrasaccharide 17 indicated that sequence of hexasaccharide 11 was correct. Mass spectrometric analysis confirmed the structural identity of Hexasaccharide 11, Pentasaccharide 16, and Tetrasaccharide 17 (FIGS. 17, 18, and 19, respectively). A m/z of 605.61[M-2H]⁻²was observed for Hexasaccharide 11 (FIG. 17). A m/z of 526.6 [M-2H]⁻² was observed for Pentasaccharide 16 (FIG. 18). A m/z of 425.1 [M-2H]⁻² was observed for Tetrasaccharide 17 (FIG. 19).

### Example 20

### Synthesis of Hexasaccharides 12 and 13 and Pentasaccharides 14 and 15

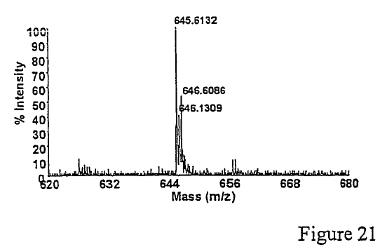

Hexasaccharide 11 (1 μg) was resuspended in 100 μl reaction buffer along with PAPS, epimerase and 2-OST1. The reaction mixture was maintained at 37° C. for 2 days. Epimerase, 2-OST1 and PAPS were added after 24 hours. The reaction mixture was passed over a Biogel P6 column (0.5 mm×200 cm) and the fractions were analyzed by LC/MS for hexasaccharide 12. The molecular ion observed for hexasaccharide 12 was 645.6 corresponding to [M-2H]⁻². The overall yield of this tandem modification step, which was calculated from MS analysis, was about 10-15%. The mass spectra of hexasaccharide 12 is shown in FIG. 21.

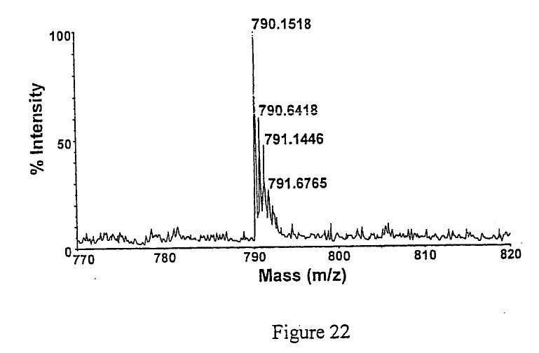

Hexasaccharide 12 was treated with 6-OST1 and 6-OST2 in 100 μl reaction buffer with PAPS. The reaction mixture was incubated at 37° C. over night and purified using a P6 column. Fractions were analyzed by LC/MS. The observed molecular ion for hexasaccharide 13 was 790.15 corresponding to [M-3H+11DBA]⁻². The mass spectra of hexasaccharide 13 is shown in FIG. 22.

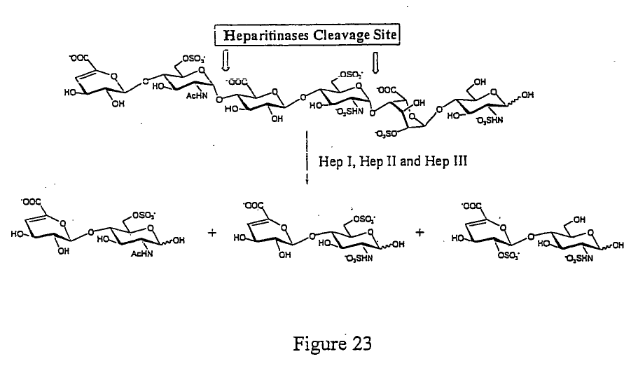

The structure of hexasaccharide 13 was derived from disaccharide analysis. Hexasaccharide 13 was treated with heparitinases to obtain disaccharides which were analyzed by LC/MS (FIG. 23). The following molecular ions were observed: 458 [M-H]⁻¹ corresponding to ΔU-GlcNAc6S; 496 [M-H]⁻¹ corresponding to ΔU-GlcNS6S and ΔU2S-GlcNS.

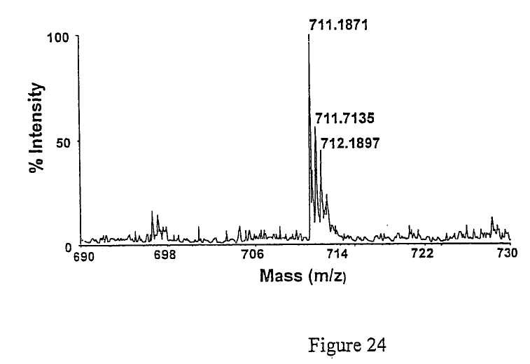

Hexasaccharide 13 was treated with Δ^{4,5}glycuronidase to remove unsaturated uronic acid at the non-reducing end, resulting in Pentasaccharide 14. The mass spectrometric analysis confirmed the formation Pentasaccharide 14 (FIG. 24). The abundant molecular ions formed adduction with ion-pairing agent, dibutylammine acetate (PBA), used in the separation by capillary HPLC. The observed m/z of 711.5, corresponding to [M-2H+1DBA]⁻², was in consistent with the calculated molecular weight.

Pentasaccharide 15 was formed by treating Pentasaccharide 14 with 3-OST1 and PAP³⁵S or PAP³⁴S and the reaction was continued overnight. The product was purified using a Biogel P6 column as outlined earlier. The radiolabeled Pentasaccharide 15 was utilized for gel mobility shift assay (GMSA) to determine its ability to bind to ATIII (FIG. 26d). Pentasaccharide 15 containing 3-O sulfate enriched with ³⁴S was analyzed by LC/MS to characterize and confirm its structure. The XIC for Pentasaccharide 15 is shown in FIG. 25A. The most abundant molecular ion was 752.1 corresponding to [M-3H+1DBA]⁻², which was in accord with the calculated molecular weight for Pentasaccharide 15. The disaccharide profile of Pentasaccharide 15 was confirmed by treating it with Heparitinases I, II and III and subsequently analyzed by mass spectroscopy (FIG. 25B).

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

### Incorporation by Reference

All publications and patent documents cited in this application are incorporated by reference in their entirety for all purposes to the same extent as if the contents of each individual publication or patent document was incorporated herein.

### Example 21

### Synthesis of a Sialyl Tn Antigen

The sialyl Tn (sTn) antigen is expressed on many tumor cell surfaces, such as colon, stomach, liver, breast and lung cancer cells. It is also expressed on gP120 HIV viral particles. Sialidases hydrolyze sTn antigens and limit their activity. Since an sTn O-glycoside has been shown to induce immune responses against various tumors, a non-hydrolyzable analog of sTn was sought, such as C-glycoside, which would have a longer in vivo half life, and serve as a vaccine for priming immune cells, and enhancing immune responses to cells and viral particles expressing sTn.

A structural comparison between sTn O-glycoside and C-glycoside antigen are demonstrated in FIG. 27. Chemical synthesis of the sTn C glycoside was accomplished as outlined in FIG. 28. As is evident from the figure, 17 steps are required for the chemical synthesis of the C-glycoside. FIG. 29 demonstrates a pathway for enzymatic synthesis of the sTn C-glycoside.

The sTn C-glycoside can be synthesized simply and efficiently via enzymatic synthesis. Further, the sTn C-glycoside can be utilized, in one embodiment, as a vaccine for prophylaxis of HIV infection and/or cancer. In another embodiment, the sTn C-glycoside can be utilized as a vaccine for prevention of HIV infection and/or cancer. Such methods and protocols are well known to one skilled in the art and may readily be applied, and include the use of in vivo and in vitro systems.

1. A method of preparing a sulfated polysaccharide capable of binding to a binding partner, comprising treating an unsulfated or incompletely sulfated polysaccharide with at least one enzyme.

2. A method of preparing heparan sulfate, comprising treating an unsulfated heparan synthon or incompletely-sulfated heparan sulfate precursor with at least one enzyme.

3. A method of preparing a sulfated polysaccharide capable of binding to a binding partner, comprising treating an unsulfated or incompletely-sulfated polysaccharide with at least one chemical reagent and at least one enzyme.

4. A method of preparing heparan sulfate, comprising treating an unsulfated heparan synthon or incompletely sulfated heparan sulfate precursor with at least one chemical reagent and at least one enzyme.

5. The method of claim 3 or 4, wherein the chemical reagent is selected from the group consisting of an N-deacetylation reagent, an N-sulfonation reagent, an epimerization reagent, and an O-sulfonation reagent.

6. The method of any of claims 1-4, wherein the enzyme is selected from the group consisting of an N-deacetylase, an N-sulfotransferase, an epimerase, and an O-sulfotransferase.

7. The method of claim 1 or 3, wherein the unsulfated or incompletely sulfated polysaccharide is treated with up to four chemical reagents.

8. The method of claim 1 or 3, wherein the unsulfated or incompletely sulfated polysaccharide is treated with up to four enzymes.

9 . The method of claim 2 or 4, wherein the heparan synthon or incompletely sulfated heparan sulfate precursor is treated with up to four chemical reagents.

10. The method of claim 2 or 4, wherein the heparan synthon or incompletely sulfated heparan sulfate precursor is treated with up to four enzymes.

11. The method of claim 1 or 3, comprising:
(a) treating an unsulfated polysaccharide with an N-deacetylating reagent;
(b) treating the step (a) product with an N-sulfating reagent;
(c) treating the step (b) product with an epimerizing reagent; and
(d) treating the step (c) product with at least one O-sulfating reagent.

12. The method of claim 2 or 4, comprising:
(a) treating a heparan synthon with an N-deacetylating reagent;
(b) treating the step (a) product with an N-sulfating reagent;
(c) treating the step (b) product with an epimerizing reagent; and
(d) treating the step (c) product with at least one O-sulfating reagent

13. The method of 2 or 4, wherein the heparan synthon is a non-sulfated N-acetyl heparosan.

14. The method of claim 1 or 3, wherein the unsulfated polysaccharide is isolated from a cell.

15. The method of claim 1 or 3 wherein the unsulfated polysaccharide is isolated from E. coli bacteria.

16. The method of claim 2 or 4, wherein the heparan synthon is isolated from a cell.

17. The method of claim 2 or 4, wherein the heparan synthon is isolated from E. coli bacteria.

18. The method of claim 11 or 12, wherein the deacetylating reagent is selected from the group consisting of a deacetylase and N-deacetylase- N-sulfotransferase.

19. The method of claim 11 or 12, wherein the deacetylating reagent is selected from the group consisting of hydrazine and a metal hydroxide.

20. The method of claim 11 or 12, wherein the N-sulfating reagent is selected from the group consisting of an N- sulfotransferase and N-deacetylase- N sulfotransferase.

21. The method of claim 11 or 12, wherein he N-sulfating reagent is selected from the group consisting of trialkylamine sulfur trioxide and chlorosulfonic acid.

22. The method of claim 11 or 12, wherein the epimerizing reagent is selected from the group consisting of C5-epimerase.

23. The method of claim 11 or 12, wherein the O-sulfating reagent incorporates a 3-O sulfate group.

24. The method of claim 11 or 12, wherein the O-sulfating reagent incorporates a 6-O sulfate group.

25. The method of claim 11 or 12, wherein-the O-sulfating reagent incorporates a 2-O sulfate group.

26. The method of claim 11 or 12, wherein the at least one O-sulfating reagent(s) incorporates one at least one of a 2-O sulfate group, a 6-O sulfate group, and a 3-O sulfate group.

27. The method of claim 11 or 12, wherein the O-sulfating reagent is a 3-O sulfotransferase.

28. The method of claim 11 or 12, wherein the 3-O sulfotransferase is selected from the group consisting of 3-OST1, 3-OST2, 3-OST3, 3-OST3a, 3-OST4, 3-OST5 and 3-OST6.

29. The method of claim 11 or 12, wherein the O-sulfating reagent is a 6-O sulfotransferase.

30. The method of claim 11 or 12, wherein the 6-O sulfotransferase is selected from the group consisting of 6-OST1, 6-OST2, and 6-OST3.

31. The method of claim 11 or 12, wherein O-sulfating reagent is a 2-O sulfotransferase.

32. A method of detecting or identifying a polysaccharide binding partner, comprising:
(a) combining the binding partner with a polysaccharide under suitable conditions for detecting a binding partner-polysaccharide interaction,
wherein the polysaccharide is prepared according to the method of claim 1; and
(b) detecting or measuring said interaction.

33. A method of determining the enzymes that participate in the synthesis of a polysaccharide, comprising treating a polysaccharide one or more of at least one N-deacetylating reagent, N-sulfating reagent, epimerizing reagent, and O-sulfating reagent and examining the enzymatic products generated by said treatment; wherein the generation of an enzymatic product is indicative that the enzyme participates in the synthesis of the polysaccharide.

34. The method of claim 33, wherein the order in which enzymes participate in the synthesis of the polysaccharide is determined.

35. An enzymatically synthesized N-sulfated, 2-O sulfated, 6-O sulfated, 3-O sulfated heparan sulfate that binds to antithrombin III.

36. An enzymatically synthesized N-sulfated, 2-O sulfated, 6-O sulfated, 3-O sulfated heparan sulfate that binds to glycoprotein D of herpes simplex virus 1, wherein the 3-O sulfation was made by 3-OST3a.

36. An enzymatically synthesized N-sulfated, 6-O sulfated, 3-O sulfated heparan sulfate that binds to antithrombin III.

37. An enzymatically synthesized Polysaccharide selected from the group consisting of Polysaccharide 1, Polysaccharide 2, Polysaccharide 3, Polysaccharide 4, Polysaccharide 5, Polysaccharide 6, Polysaccharide 7, Polysaccharide 8, Polysaccharide 9, Polysaccharide 10, Hexasaccharide 11, Hexasaccharide 12, Hexasaccharide 13, Pentasaccharide 14, Pentasaccharide 15, Pentasaccharide 16, and Tetrasaccharide 17.

38. The method of claim 37, wherein the polysaccharide is a functional analogue or derivative of a pentasaccharide.

39. A method of synthesizing a selected from the group consisting of Polysaccharide 1, Polysaccharide 2, Polysaccharide 3, Polysaccharide 4, Polysaccharide 5, Polysaccharide 6, Polysaccharide 7, Polysaccharide 8, Polysaccharide 9, Polysaccharide 10, Hexasaccharide 11, Hexasaccharide 12, Hexasaccharide 13, Pentasaccharide 14, Pentasaccharide 15, Pentasaccharide 16, and Tetrasaccharide 17.

40. A method of purifying a polysaccharide comprising the steps of isolating the polysaccharide using mass spectrometry.

41. The method of claim 40, wherein the Polysaccharide is selected from the group consisting of Polysaccharide 1, Polysaccharide 2, Polysaccharide 3, Polysaccharide 4, Polysaccharide 5, Polysaccharide 6, Polysaccharide 7, Polysaccharide 8, Polysaccharide 9, Polysaccharide 10, Hexasaccharide 11, Hexasaccharide 12, Hexasaccharide 13, Pentasaccharide 14, Pentasaccharide 15, Pentasaccharide 16, and Tetrasaccharide 17.

42. A method of 2-O-sulfating Polysaccharide 10, comprising the steps of treating Polysaccharide 10 with epimerase and 2-OST1.

43. The method of claim 1 or 3, comprising the steps of
(a) treating a heparan synthon with an N-sulfating reagent;
(b) treating the step (a) product with a heparitinase reagent;
(c) treating the step (b) product with an epimerizing reagent and a 2-O-sulfating reagent;
(d) treating the step (c) product with a 6-O-sulfating reagent;
(e) treating the step (d) product with a Δ^{4,5} glycuronidase; and
(f) treating the step (e) product with 3-O-sulfating reagent.

44. The method of claim 1 or 3, comprising the steps of
(a) treating an unsulfated polysaccharide with an N-sulfating reagent;
(b) treating the step (a) product with an epimerizing reagent;
(c) treating the step (b) product with a 6-O-sulfating reagent; and
(d) treating the step (c) product with nitrous acid and sodium borohydride.

45. The method of claim 43 or 44, wherein the products of step (d) are disaccharides.

46. The method of claim 1 or 3, comprising the steps of
(a) treating an unsulfated polysaccharide with an N-sulfating reagent; and
(b) treating the step (a) product with an epimerizing reagent and an 2-O-sulfating reagent.

47. The method of claim 1 or 3, comprising the steps of
(a) treating an unsulfated polysaccharide with an N-sulfating reagent;
(b) treating the step (a) product with an epimerizing reagent; and
(c) treating the step (b) product with a 2-O-sulfating reagent.

48. The method of claim 1 or 3, comprising the steps of
(a) treating an unsulfated polysaccharide with an N-sulfating reagent; and
(b) treating the step (a) product with an 2-O-sulfating reagent.

49. The method of determining the structural identification of Hexasaccharide 11, comprising the steps of
(a) treating Hexasaccharide 11 with A ^{4,5}-β-glycuronidase; and
(b) treating the step (a) product with an α-N-Acetylglucosaminidase.

50. A method of producing Polysaccharide 2, the method comprising the step of treating Polysaccharide 1 with an N-sulfating reagent.

51. A method of producing Polysaccharide 3, the method comprising the step of treating Polysaccharide 2 with an epimerizing agent.

52. A method of producing Polysaccharide 4, the method comprising the step of treating Polysaccharide 3 with a 2-O-sulfating reagent.

53. A method of producing Polysaccharide 5, the method comprising the step of treating Polysaccharide 4 with a 6-O-sulfating reagent.

54. A method of producing Polysaccharide 6, the method comprising the step of treating Polysaccharide 5 with a 3-O-sulfating reagent.

55. A method of producing Polysaccharide 7, the method comprising the step of treating Polysaccharide 3 with a 6-O-sulfating reagent.

56. A method of producing Polysaccharide 8, the method comprising the step of treating Polysaccharide 7 with a 3-O-sulfating reagent;

57. A method of producing Polysaccharide 9, the method comprising the step of treating Polysaccharide 5 with a 3-O-sulfating reagent.

58. A method of producing Polysaccharide 10, the method comprising the step of treating Polysaccharide 1 with an N-sulfating reagent.

59. A method of producing Hexasaccharide 11, the method comprising the step of treating Polysaccharide 10 with a heparitinase reagent.

60. A method of producing Hexasaccharide 12, the method comprising the step of treating Hexasaccharide 11 with an epimerase reagent and a 2-O-sulfating reagent

61. A method of producing Hexasaccharide 13, the method comprising the step of treating Hexasaccharide 12 with a 6-O-sulfating reagent.

62. A method of producing Pentasaccharide 14, the method comprising the step of treating Hexasaccharide 13 with Δ^{4,5}-β-glycuronidase reagent.

63. A method of producing Pentasaccharide 15, the method comprising the step of treating Pentasaccharide 14 with a 3-O-sulfating reagent.

64. A method of producing Pentasaccharide 16, the method comprising the step of treating Pentasaccharide 11 with a Δ^{4,5}-β-glycuronidase reagent.

65. A method of producing Tetrasaccharide 17, the method comprising the step of treating Pentasaccharide 16 with a α-N-Acetylglucosaminidase.
